(19) United States
(12) Patent Application Publication
(10) Pub. No.: US 2017/0200902 A1
(43) Pub. Date: Jul. 13, 2017

(54) COMPOUND, ORGANIC OPTOELECTRONIC DIODE CONTAINING SAME, AND DISPLAY DEVICE

(71) Applicant: SAMSUNG SDI CO., LTD., Yongin-si, Gyeonggi-do (KR)

(72) Inventors: Seung-Jae LEE, Suwon-si, Gyeonggi-do (KR); Byung-Ku KIM, Suwon-si, Gyeonggi-do (KR); Young-Kwon KIM, Suwon-si, Gyeonggi-do (KR); Soo-Hyun MIN, Suwon-si, Gyeonggi-do (KR); Joo-Hee SEO, Suwon-si, Gyeonggi-do (KR); Eun-Sun YU, Suwon-si, Gyeonggi-do (KR)

(21) Appl. No.: 15/316,720

(22) PCT Filed: Aug. 3, 2015
(86) PCT No.: PCT/KR2015/008105

§ 371 Date: Dec. 6, 2016

(30) Foreign Application Priority Data
Aug. 12, 2014 (KR) 10-2014-0104458

(51) Int. Cl. H01L 51/00; C07D 239/26; C07D 213/16; C09K 11/02; C07D 251/24

(52) U.S. Cl. CPC H01L 51/0067; C09K 11/025; C07D 251/24; C07D 213/16; C07D 239/26; H01L 51/5012

(57) ABSTRACT

A compound represented by the following Chemical Formula 1, an organic optoelectric device including the same and a display device including the organic optoelectric device are disclosed. The detailed descriptions of Chemical Formula 1 are the same as defined in the specification.

### TECHNICAL FIELD

A compound, an organic optoelectric device, and a display device are disclosed.

### BACKGROUND ART

An organic optoelectric device is a device that converts electrical energy into photoenergy, and vice versa.

An organic optoelectric device may be classified as follows in accordance with its driving principles. One is an optoelectric device where excitons are generated by photoenergy, separated into electrons and holes, and are transferred to different electrodes to generate electrical energy, and the other is a light emitting device where a voltage or a current is supplied to an electrode to generate photoenergy from electrical energy.

Examples of an organic optoelectric device may be an organic photoelectric device, an organic light emitting diode, an organic solar cell and an organic photo conductor drum.

Of these, an organic light emitting diode (OLED) has recently drawn attention due to an increase in demand for flat panel displays. Such an organic light emitting diode converts electrical energy into light by applying current to an organic light emitting material. It has a structure in which an organic layer is interposed between an anode and a cathode. Herein, an organic layer may include an emission layer and optionally an auxiliary layer, and the auxiliary layer may include, for example at least one selected from a hole injection layer, a hole transport layer, an electron blocking layer, an electron transport layer, an electron injection layer and a hole blocking layer in order increase efficiency and stability of an organic light emitting diode.

Performance of an organic light emitting diode may be affected by characteristics of the organic layer, and among them, may be mainly affected by characteristics of an organic material of the organic layer.

Particularly, development for an organic material being capable of increasing hole and electron mobility and simultaneously increasing electrochemical stability is needed so that the organic light emitting diode may be applied to a large-size flat panel display.

### DISCLOSURE

### Technical Problem

One embodiment provides a compound being capable of realizing an organic optoelectric device having high efficiency and long life-span.

Another embodiment provides an organic optoelectric device including the compound.

Yet another embodiment provides a display device including the organic optoelectric device.

### Technical Solution

In one embodiment of the present invention, a compound represented by Chemical Formula 1 is provided.

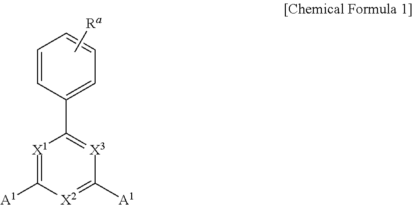

In Chemical Formula 1,

X¹ to X³ are independently N or CR^{b},

at least one of X¹ to X³ is N,

R^{a} and R^{b} are each independently hydrogen, deuterium, or a substituted or unsubstituted C1 to C10 alkyl group, and

A¹ is represented by Chemical Formula I or II,

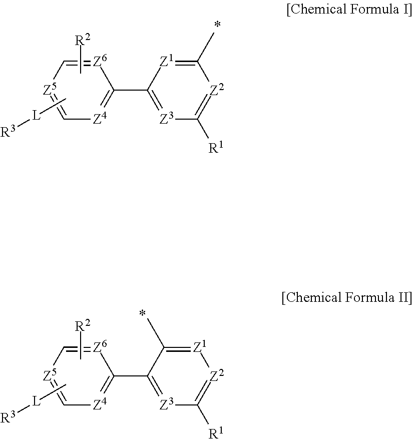

wherein, in Chemical Formulae I and II,

Z¹ to Z⁶ are each independently N, C or CR^{c},

R¹, R² and R^{c} are each independently hydrogen, deuterium, a substituted or unsubstituted C1 to C30 alkyl group, a substituted or unsubstituted C3 to C30 cycloalkyl group, a substituted or unsubstituted C2 to C30 heteroaryl group, a substituted or unsubstituted C6 to C30 aryl group, a substituted or unsubstituted C6 to C30 arylamine group, a substituted or unsubstituted C1 to C30 alkoxy group, a substituted or unsubstituted C3 to C40 silyl group, a halogen, a halogen-containing group, a cyano group, a hydroxyl group, an amino group, a nitro group, a carboxyl group, a ferrocenyl group, or a combination thereof,

L is a single bond, a C6 to C30 arylene group, or a C2 to C30 heterocyclic group,

R³ is hydrogen, a substituted or unsubstituted C6 to C30 aryl group, or a substituted or unsubstituted nitrogen-containing C2 to C30 heterocyclic group except a carbazolyl group,

when the L is a single bond, at least one of R¹ to R³ is not hydrogen, and

* is a linking point,

wherein “substituted” refers to that at least one hydrogen is replaced by deuterium, a halogen, a hydroxy group, an amino group, a C1 to C30 alkyl group, a C6 to C30 aryl group, or a C2 to C30 heteroaryl group.

The compound according to one embodiment of the present invention may be used for an organic optoelectric device.

In another embodiment of the present invention, an organic optoelectric device includes an anode and a cathode facing each other and at least one organic layer between the anode and the cathode, wherein the organic layer includes an emission layer and at least one auxiliary layer selected from a hole injection layer, a hole transport layer, an electron blocking layer, an electron transport layer, an electron injection layer, and a hole blocking layer, and the auxiliary layer includes the compound.

In yet another embodiment of the present invention, a display device including the organic optoelectric device is provided.

### Advantageous Effects

An organic optoelectric device having high efficiency and long life-span may be realized.

### DESCRIPTION OF THE DRAWINGS

FIGS. 1 and 2 are cross-sectional views showing organic light emitting diodes according to one embodiment of the present invention.

### BEST MODE

Hereinafter, embodiments of the present invention are described in detail. However, these embodiments are exemplary, the present invention is not limited thereto and the present invention is defined by the scope of claims.

In the present specification, when a definition is not otherwise provided, the term “substituted” refers to one substituted with a deuterium, a halogen, a hydroxy group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C3 to C30 cycloalkyl group, a C3 to C30 heterocycloalkyl group, a C6 to C30 aryl group, a C6 to C30 heteroaryl group, a C1 to C20 alkoxy group, a fluoro group, a C1 to C10 trifluoroalkyl group such as a trifluoromethyl group, or a cyano group, instead of at least one hydrogen of a substituent or a compound.

In the present specification, when specific definition is not otherwise provided, “hetero” refers to one including 1 to 3 hetero atoms selected from N, O, S, P, and Si, and remaining carbons in one functional group.

In the present specification, when a definition is not otherwise provided, “alkyl group” refers to an aliphatic hydrocarbon group. The alkyl group may be “a saturated alkyl group” without any double bond or triple bond.

The alkyl group may be a C1 to C20 alkyl group. More specifically, the alkyl group may be a C1 to C10 alkyl group or a C1 to C6 alkyl group. For example, a C1 to C4 alkyl group may have 1 to 4 carbon atoms in an alkyl chain which may be selected from methyl, ethyl, propyl, iso-propyl, n-butyl, iso-butyl, sec-butyl, and t-butyl.

Specific examples of the alkyl group may be a methyl group, an ethyl group, a propyl group, an isopropyl group, a butyl group, an isobutyl group, a t-butyl group, a pentyl group, a hexyl group, a cyclopropyl group, a cyclobutyl group, a cyclopentyl group, a cyclohexyl group, and the like.

In the present specification, the term “aryl group” refers to a substituent including all element of the cycle having p-orbitals which form conjugation, and may be monocyclic, polycyclic or fused ring polycyclic (i.e., rings sharing adjacent pairs of carbon atoms) functional group.

In the present specification, the term “heterocyclic group” refers to a cyclic compound such as an aryl group, a cycloalkyl group, a fused ring thereof, or a combination thereof including at least one heteroatoms selected from N, O, S, P, and Si, and remaining carbons. When the heterocyclic group is a fused ring, the entire ring or each ring of the heterocyclic group may include one or more heteroatoms. Accordingly, the heterocyclic group is a general term including a heteroaryl group.

More specifically, the substituted or unsubstituted C6 to C30 aryl group and/or the substituted or unsubstituted C2 to C30 heterocyclic group may be a substituted or unsubstituted phenyl group, a substituted or unsubstituted naphthyl group, a substituted or unsubstituted anthracenyl group, a substituted or unsubstituted phenanthrylene group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted chrysenyl group, a substituted or unsubstituted triphenylenyl group, a substituted or unsubstituted perylenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridinyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted fluorenyl group, a substituted or unsubstituted carbazolyl group, a substituted or unsubstituted dibenzofuranyl group, a substituted or unsubstituted dibenzothiophenyl group, a combination thereof, or a combined fused ring of the foregoing groups, but are not limited thereto.

In the present specification, the substituted or unsubstituted nitrogen-containing C2 to C30 heterocyclic group except a carbazolyl group refers to a substituted or unsubstituted imidazolyl group, a substituted or unsubstituted triazolyl group, a substituted or unsubstituted tetrazolyl group, a substituted or unsubstituted oxadiazolyl group, a substituted or unsubstituted oxatriazolyl group, a substituted or unsubstituted thiatriazolyl group, a substituted or unsubstituted benzimidazolyl group, a substituted or unsubstituted benzotriazolyl group, a substituted or unsubstituted pyridinyl group, a substituted or unsubstituted pyrimidinyl group, a substituted or unsubstituted triazinyl group, a substituted or unsubstituted pyrazinyl group, a substituted or unsubstituted pyridazinyl group, a substituted or unsubstituted purinyl group, a substituted or unsubstituted quinolinyl group, a substituted or unsubstituted isoquinolinyl group, a substituted or unsubstituted phthalazinyl group, a substituted or unsubstituted naphpyridinyl group, a substituted or unsubstituted quinoxalinyl group, a substituted or unsubstituted quinazolinyl group, a substituted or unsubstituted acridinyl group, a substituted or unsubstituted phenanthrolinyl group, a substituted or unsubstituted phenazinyl group, or a combination thereof.

In the present specification, the single bond may refer to direct linkage without carbon a hetero atom except carbon, and specifically when L is a single bond, a substituent linked to L directly links to core directly. That is to say, in the present specification, a single bond excludes methylene including carbon, and the like.

In the specification, hole characteristics refer to characteristics capable of donating an electron when an electric field is applied and that a hole formed in the anode is easily injected into the emission layer and transported in the emission layer due to conductive characteristics according to highest occupied molecular orbital (HOMO) level.

In addition, electron characteristics refer to characteristics capable of accepting an electron when an electric field is applied and that an electron formed in the cathode is easily injected into the emission layer and transported in the emission layer due to conductive characteristics according to lowest unoccupied molecular orbital (LUMO) level.

Hereinafter, a compound according to one embodiment is described.

In one embodiment of the present invention, a compound represented by Chemical Formula 1 is provided.

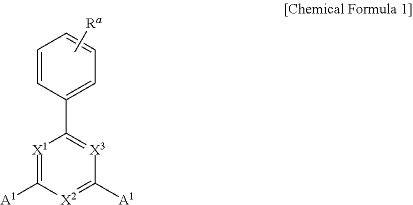

In Chemical Formula 1,

X¹ to X³ are independently N or CR^{b},

at least one of X¹ to X³ is N,

R^{a} and R^{b} are each independently hydrogen, deuterium, or a substituted or unsubstituted C1 to C10 alkyl group, and

A¹ is represented by Chemical Formula I or II,

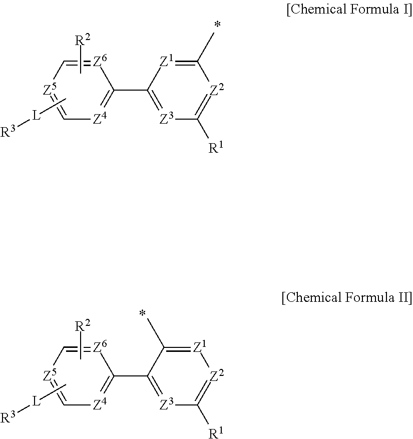

in Chemical Formulae I and II,

Z¹ to Z⁶ are each independently N, C or CR^{C},

R¹, R² and R^{c} are each independently hydrogen, deuterium, a substituted or unsubstituted C1 to C30 alkyl group, a substituted or unsubstituted C3 to C30 cycloalkyl group, a substituted or unsubstituted C2 to C30 heteroaryl group, a substituted or unsubstituted C6 to C30 aryl group, a substituted or unsubstituted C6 to C30 arylamine group, a substituted or unsubstituted C1 to C30 alkoxy group, a substituted or unsubstituted C3 to C40 silyl group, a halogen, a halogen-containing group, a cyano group, a hydroxyl group, an amino group, a nitro group, a carboxyl group, a ferrocenyl group, or a combination thereof,

L is a single bond, a C6 to C30 arylene group, or a C2 to C30 heterocyclic group,

R³ is hydrogen, a substituted or unsubstituted C6 to C30 aryl group, or a substituted or unsubstituted nitrogen-containing C2 to C30 heterocyclic group except a carbazolyl group,

when the Lisa single bond, at least one of R¹ to R³ is not hydrogen, and

* is a linking point,

wherein “substituted” refers to that at least one hydrogen is replaced by deuterium, a halogen, a hydroxy group, an amino group, a C1 to C30 alkyl group, a C6 to C30 aryl group, or a C2 to C30 heteroaryl group.

The compound represented by Chemical Formula 1 includes the same substituents forming a biaxial symmetry with a core of a heteroaryl group containing at least one nitrogen as its center.

The substituent forming a biaxial symmetry may be bonded at a meta or ortho position with the core.

The compound includes at least one nitrogen-containing ring and thus, may have a structure easily accepting the electrons when an electric field is applied thereto and accordingly, decrease a driving voltage of an organic optoelectric device manufactured by using the compound.

In addition, the compound includes the same substituents forming a biaxial symmetry and thus is easily and fast synthesized through small steps and also, becomes more crystalline and thus, has high purity by easily removing impurities.

The compound has a smaller molecular weight than a compound having a three-branched structure and thus, may have a structure having a desired HOMO, LUMO and T₁ through connection to various substituents and a low deposition temperature.

In particular, since the substituents are connected to the core at a meta position or an ortho position, a life-span may be improved by separating an electron cloud of HOMO and LUMO and thus, smoothing a flow of holes and electrons. In addition, when the substituents are connected at a meta or ortho position rather than a para position, the compound may have a low deposition temperature.

On the other hand, the substituents are connected at a para position and thus, forms a flat structure, this flat structure shows good thin film characteristics and thus, has a packing effect during the deposition, and resultantly, the packed film may bring about a negative influence on life-span of a device.

Accordingly, when the compound having a bond at a meta position or an ortho position according to one embodiment of the present invention is applied to an organic optoelectric device, the organic optoelectric device may have high efficiency, a long life-span, and characteristics of being driven at a low voltage.

The above Chemical Formula 1 may be expressed as one of the following Chemical Formulae I-a, I-b, I-c, II-a, II-b and II-c depending on a bonding position of a terminal substituent.

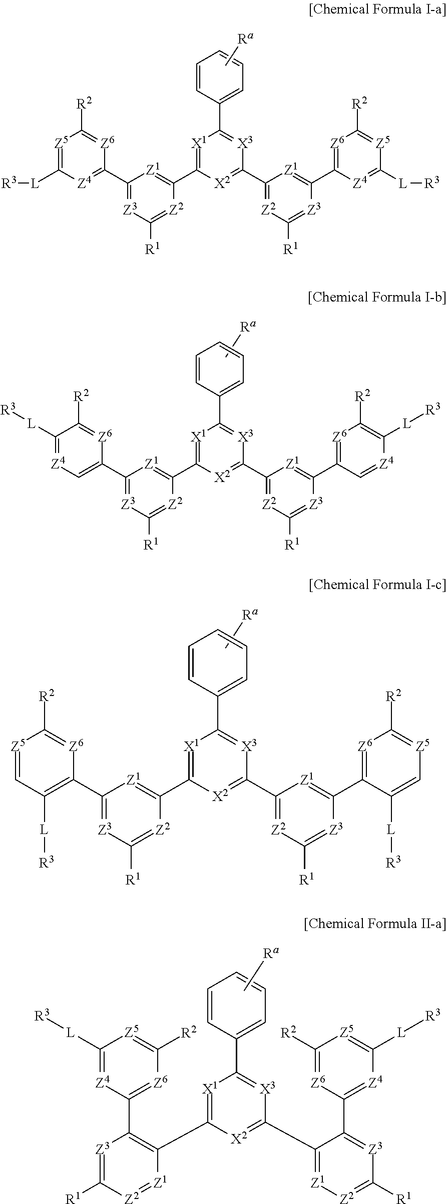

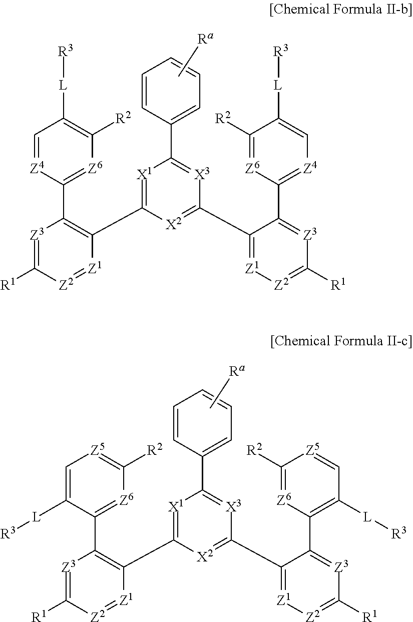

In Chemical Formulae I-a, I-b, I-c, II-a, II-b and II-c,

X¹ to X³, R^{a}, R^{b}, Z¹ to Z⁶, R¹, R², R^{c}, L and R³ are the same as described above.

In the substituents represented by Chemical Formula I or Chemical Formula II that belongs to Chemical Formula 1, Z¹ to Z⁶ may be all carbon, or may include N. Specifically, they may be represented by one of Chemical Formula I-d, I-e, I-f, II-d and II-e.

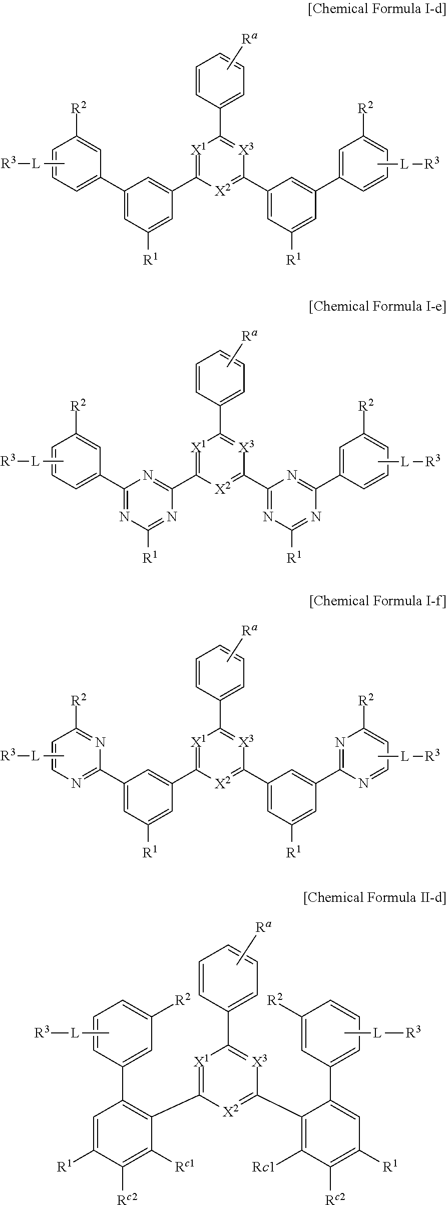

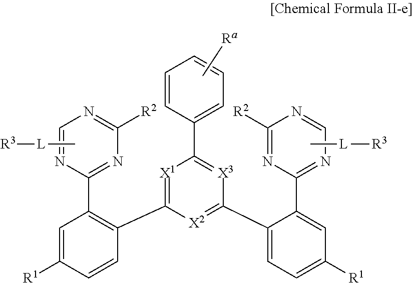

In Chemical Formulae I-d, I-e, I-f, II-d and II-e,

X¹ to X³, R^{a}, R^{b}, R¹, R², L and R³ are the same as defined above, and

R^{c1} and R^{c2} are the same as R¹ defined above.

In the definition of the R³, the substituted or unsubstituted nitrogen-containing C2 to C30 heterocyclic group except a carbazolyl group refers to a substituent having characteristics to accept electrons, when an electric field is applied and having characteristics to inject electrons formed in the cathode easily into the emission layer and to transport into the emission layer due to conductive characteristics according to lowest unoccupied molecular orbital (LUMO) level, and may be, for example a substituted or unsubstituted imidazolyl group, a substituted or unsubstituted triazolyl group, a substituted or unsubstituted tetrazolyl group, a substituted or unsubstituted oxadiazolyl group, a substituted or unsubstituted oxatriazolyl group, a substituted or unsubstituted thiatriazolyl group, a substituted or unsubstituted benzimidazolyl group, a substituted or unsubstituted benzotriazolyl group, a substituted or unsubstituted pyridinyl group, a substituted or unsubstituted pyrimidinyl group, a substituted or unsubstituted triazinyl group, a substituted or unsubstituted pyrazinyl group, a substituted or unsubstituted pyridazinyl group, a substituted or unsubstituted purinyl group, a substituted or unsubstituted quinolinyl group, a substituted or unsubstituted isoquinolinyl group, a substituted or unsubstituted phthalazinyl group, a substituted or unsubstituted naphpyridinyl group, a substituted or unsubstituted quinoxalinyl group, a substituted or unsubstituted quinazolinyl group, a substituted or unsubstituted acridinyl group, a substituted or unsubstituted phenanthrolinyl group, a substituted or unsubstituted phenazinyl group, or a combination thereof.

The R¹, R^{c1}, R^{c2}, R² and R^{c} may be each independently hydrogen, a substituted or unsubstituted phenyl group, a substituted or unsubstituted biphenyl group, a substituted or unsubstituted terphenyl group, a substituted or unsubstituted naphthyl group, a substituted or unsubstituted pyridinyl group, a substituted or unsubstituted pyrimidinyl group, or substituted or triazinyl group, and

the R³ is hydrogen, a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted triphenylene group, a substituted or unsubstituted imidazolyl group, a substituted or unsubstituted triazolyl group, a substituted or unsubstituted tetrazolyl group, a substituted or unsubstituted oxadiazolyl group, a substituted or unsubstituted oxatriazolyl group, a substituted or unsubstituted thiatriazolyl group, a substituted or unsubstituted benzimidazolyl group, a substituted or unsubstituted benzotriazolyl group, a substituted or unsubstituted pyridinyl group, a substituted or unsubstituted pyrimidinyl group, a substituted or unsubstituted triazinyl group, a substituted or unsubstituted pyrazinyl group, a substituted or unsubstituted pyridazinyl group, a substituted or unsubstituted purinyl group, a substituted or unsubstituted quinolinyl group, a substituted or unsubstituted isoquinolinyl group, a substituted or unsubstituted phthalazinyl group, a substituted or unsubstituted naphpyridinyl group, a substituted or unsubstituted quinoxalinyl group, a substituted or unsubstituted quinazolinyl group, a substituted or unsubstituted acridinyl group, a substituted or unsubstituted azaphenanthrenyl group, a substituted or unsubstituted phenanthrolinyl group, a substituted or unsubstituted phenazinyl group, or a combination thereof.

Specifically, the R³ may be selected from substituted or unsubstituted groups of Group I.

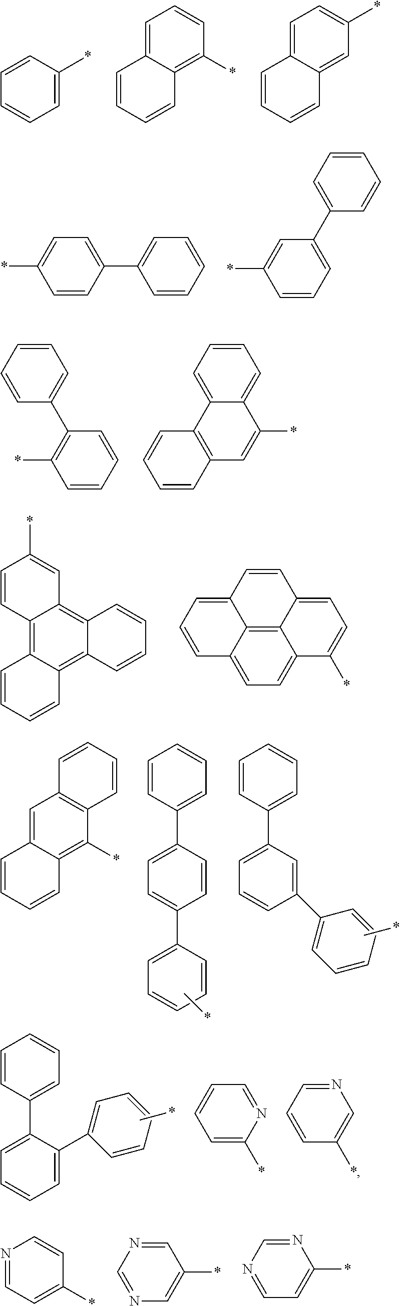

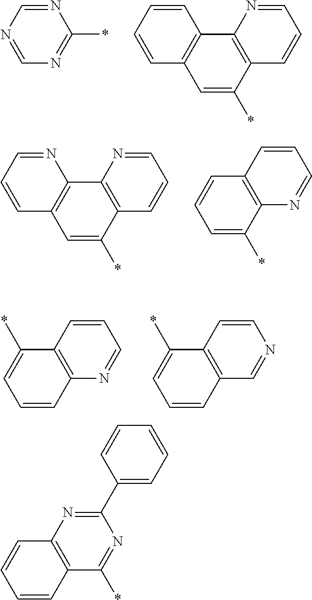

In Group I,

* is a linking point.

Herein “substituted” refers to that at least one hydrogen is replaced by deuterium, a halogen, a hydroxy group, an amino group, a C1 to C30 alkyl group, a C6 to C30 aryl group, or a C2 to C30 heteroaryl group.

The L may be specifically a single bond, a substituted or unsubstituted phenylene group, a substituted or unsubstituted biphenylene group, a substituted or unsubstituted naphthyl group, a substituted or unsubstituted pyridinyl group, a substituted or unsubstituted pyrimidyl group, a substituted or unsubstituted triazinyl group, or a combination thereof. For example, the L may be selected from substituted or unsubstituted groups of Group II.

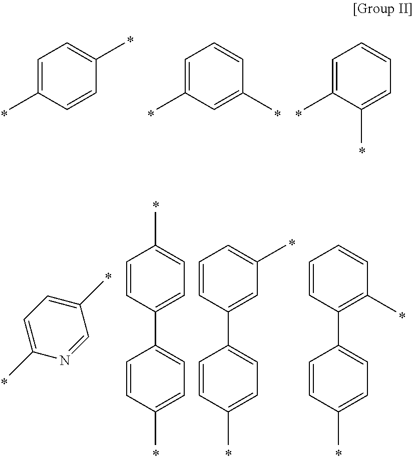

In Group II,

* is a linking point.

Herein “substituted” refers to that at least one hydrogen is replaced by deuterium, a halogen, a hydroxy group, an amino group, a C1 to C30 alkyl group, a C6 to C30 aryl group, or a C2 to C30 heteroaryl group.

The compound represented by Chemical Formula 1 may be, for example the following compounds, but is not limited thereto.

In the following specific chemical formulae, heteroatoms are all “N”.

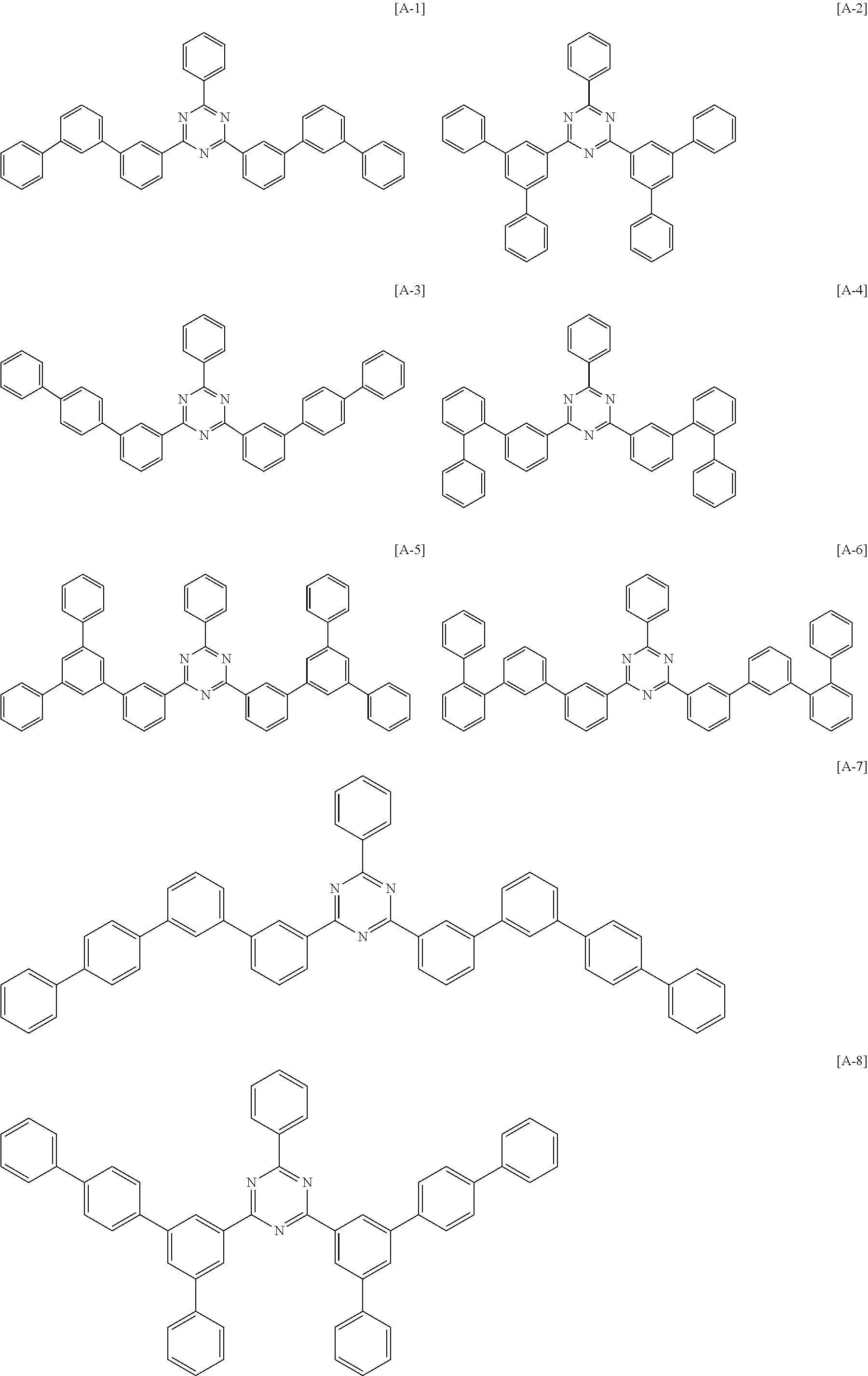

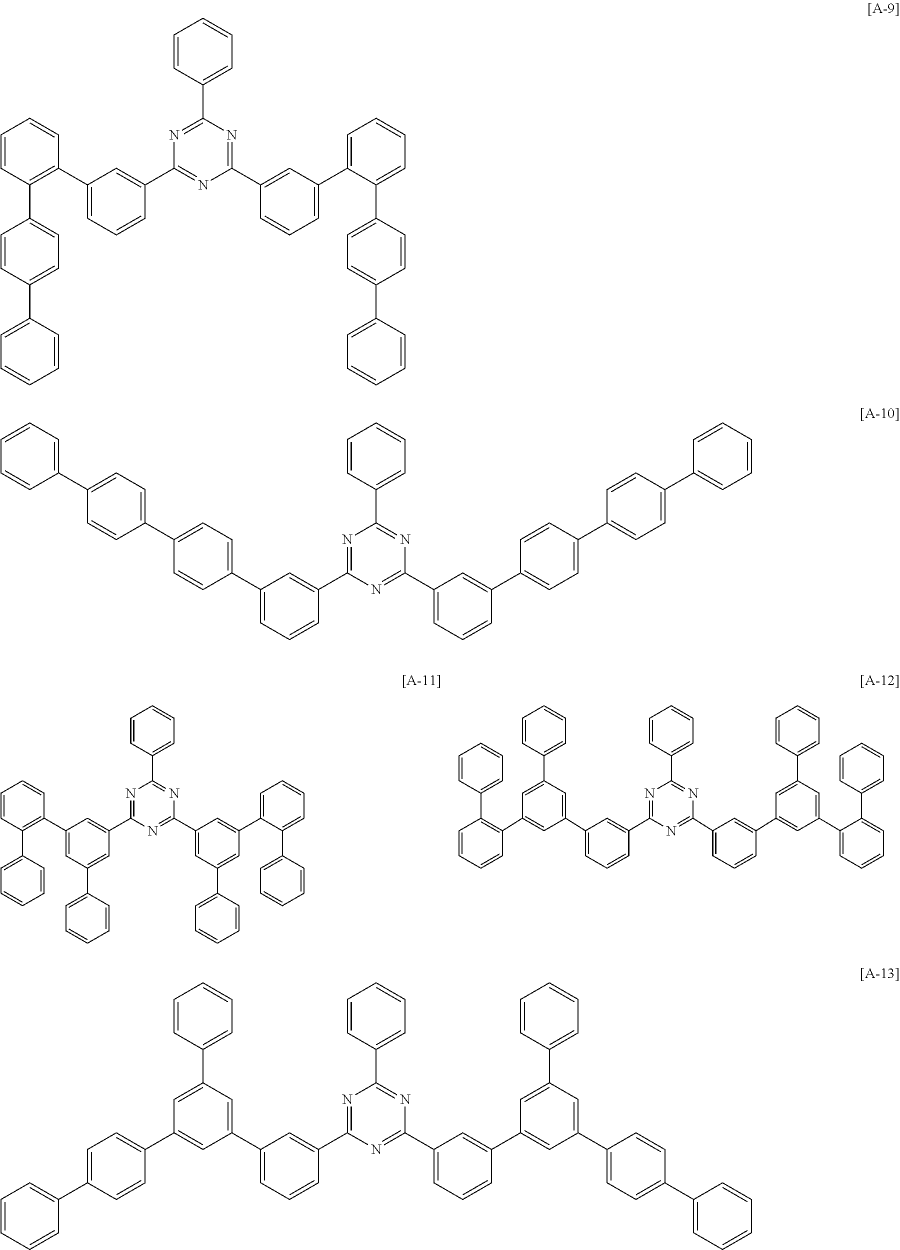

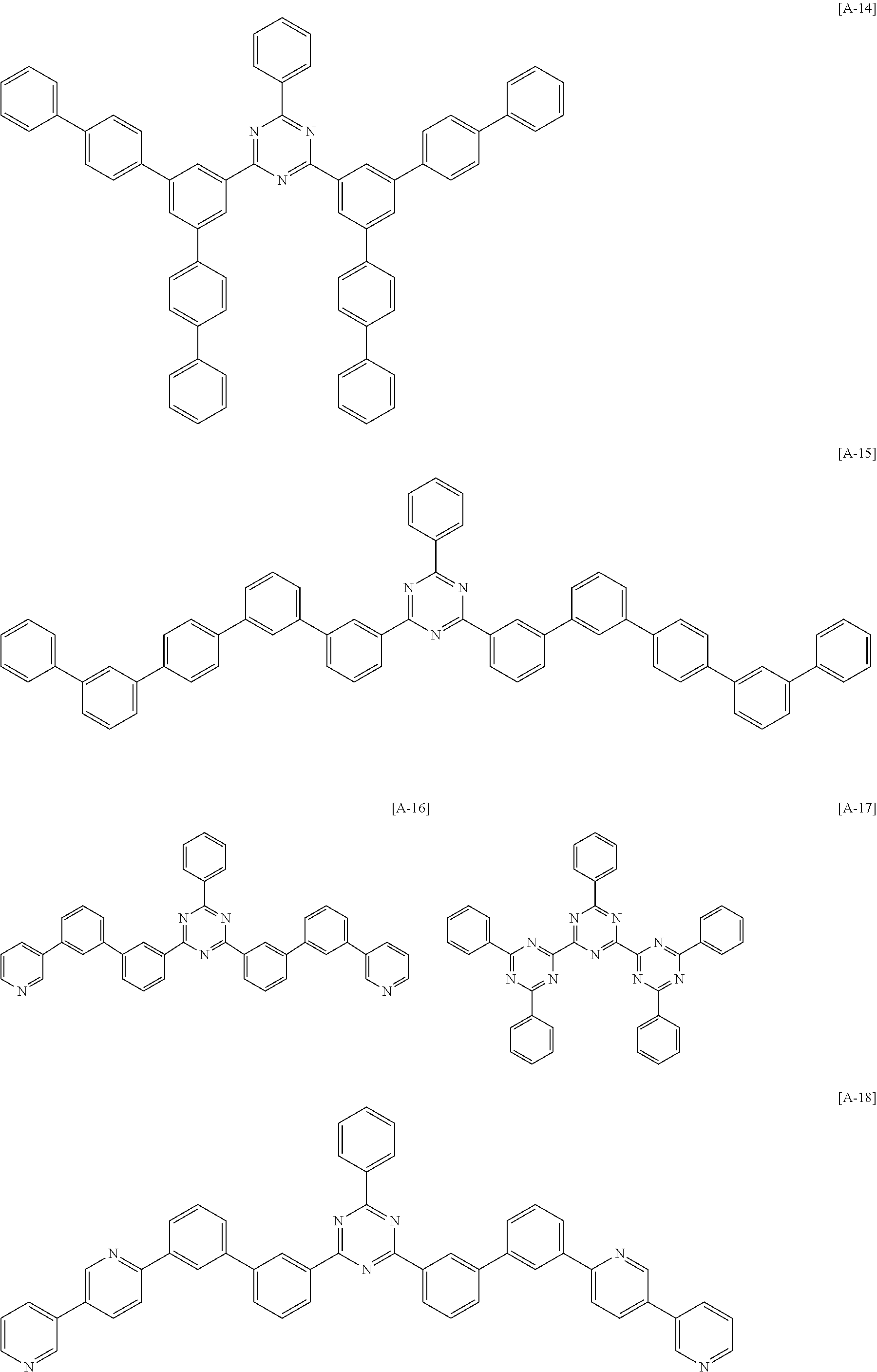

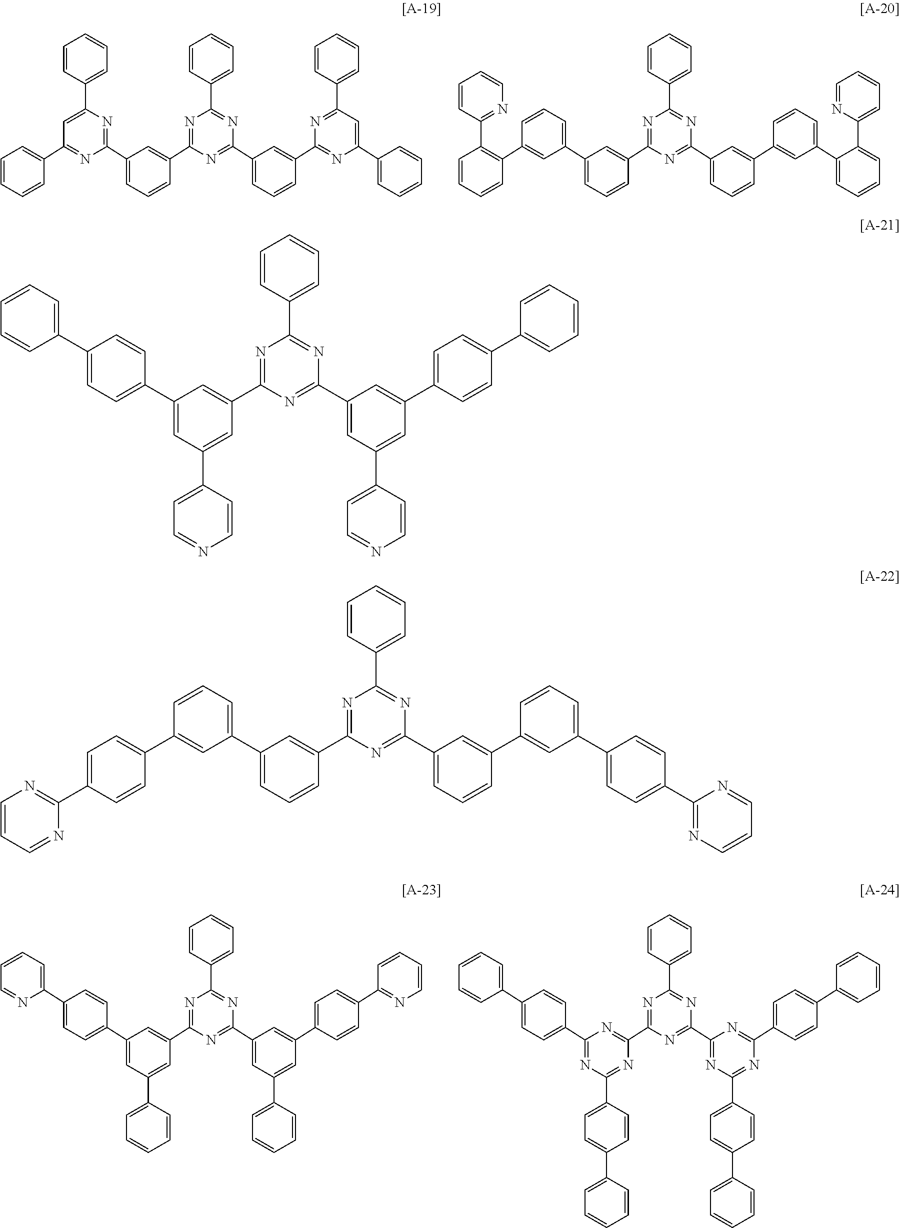

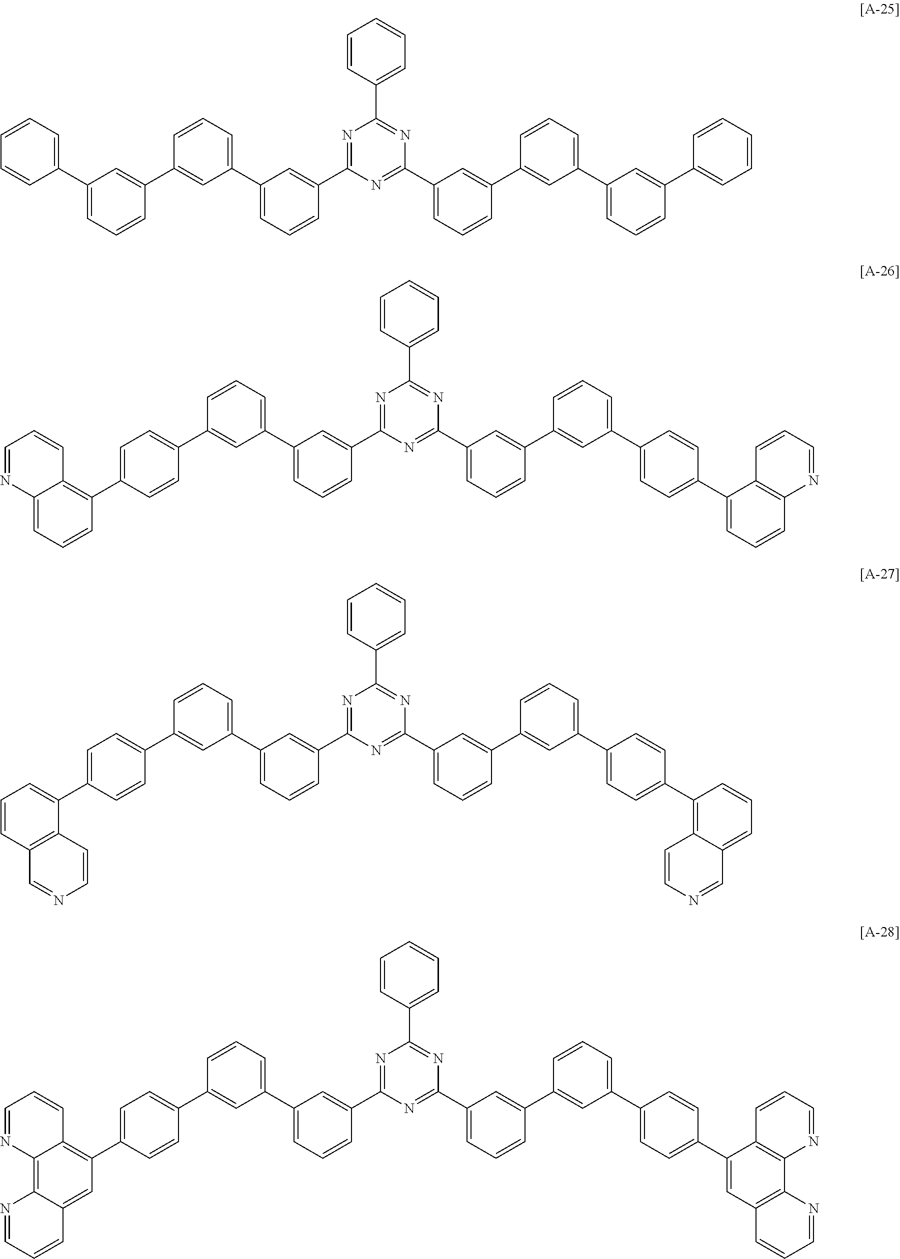 [SMILES: c1ccc(-c2cccc(-c3cccc(-c4cccc(-c5nc(-c6ccccc6)nc(-c6cccc(-c7cccc(-c8cccc(-c9ccccc9)c8)c7)c6)n5)c4)c3)c2)cc1 ; c1ccc(-c2nc(-c3cccc(-c4cccc(-c5ccc(-c6cccc7ncccc67)cc5)c4)c3)nc(-c3cccc(-c4cccc(-c5ccc(-c6cccc7ncccc67)cc5)c4)c3)n2)cc1 ; c1ccc(-c2nc(-c3cccc(-c4cccc(-c5ccc(-c6cccc7cnccc67)cc5)c4)c3)nc(-c3cccc(-c4cccc(-c5ccc(-c6cccc7cnccc67)cc5)c4)c3)n2)cc1 ; c1ccc(-c2nc(-c3cccc(-c4cccc(-c5ccc(-c6cc7cccnc7c7ncccc67)cc5)c4)c3)nc(-c3cccc(-c4cccc(-c5ccc(-c6cc7cccnc7c7ncccc67)cc5)c4)c3)n2)cc1]

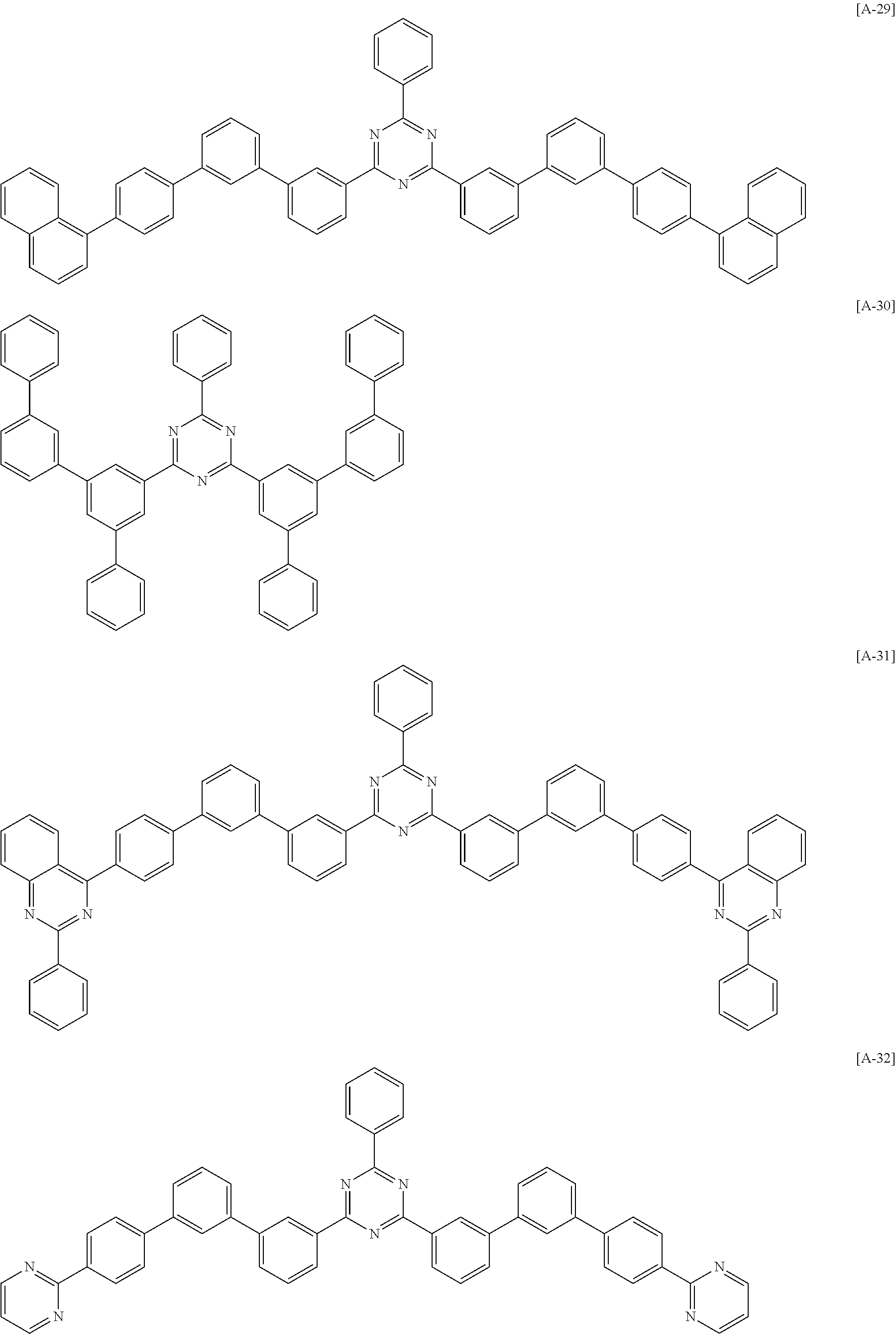

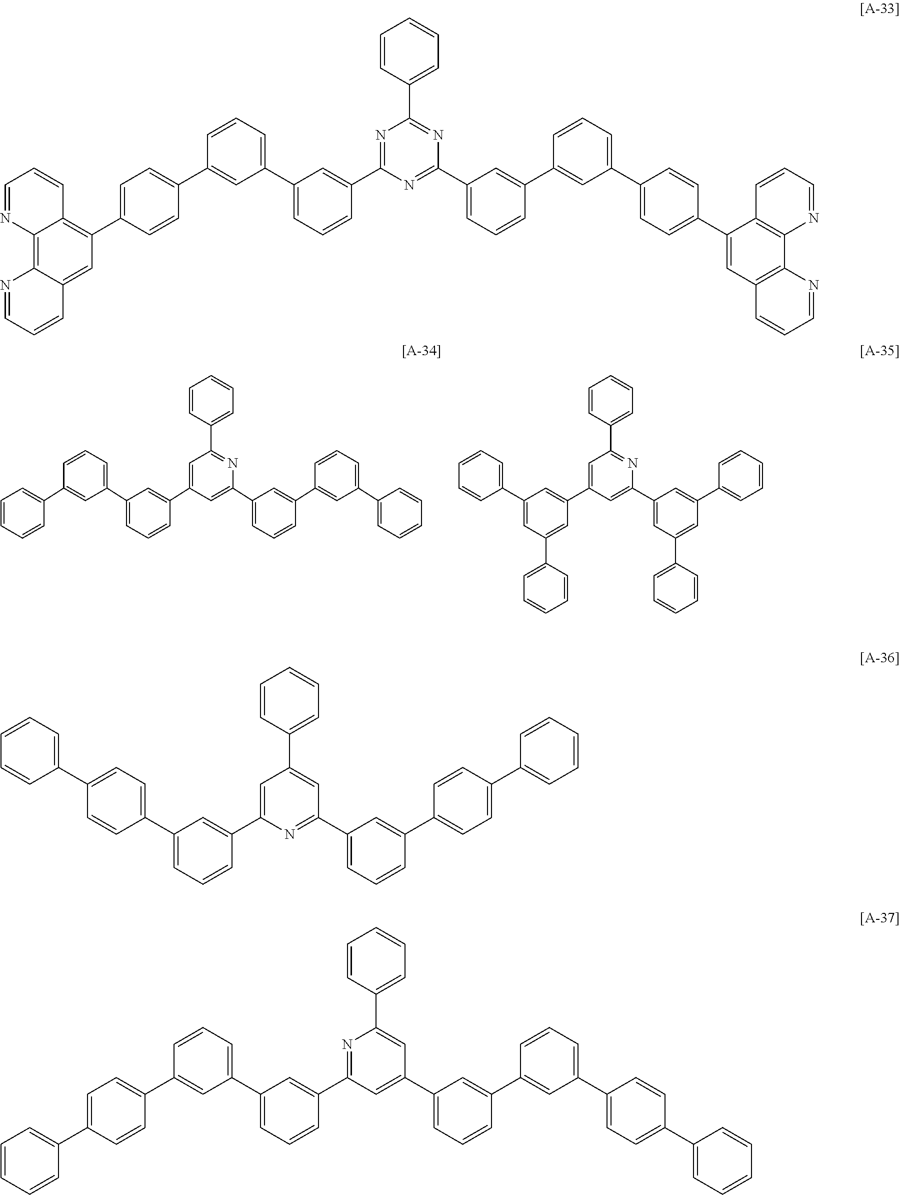

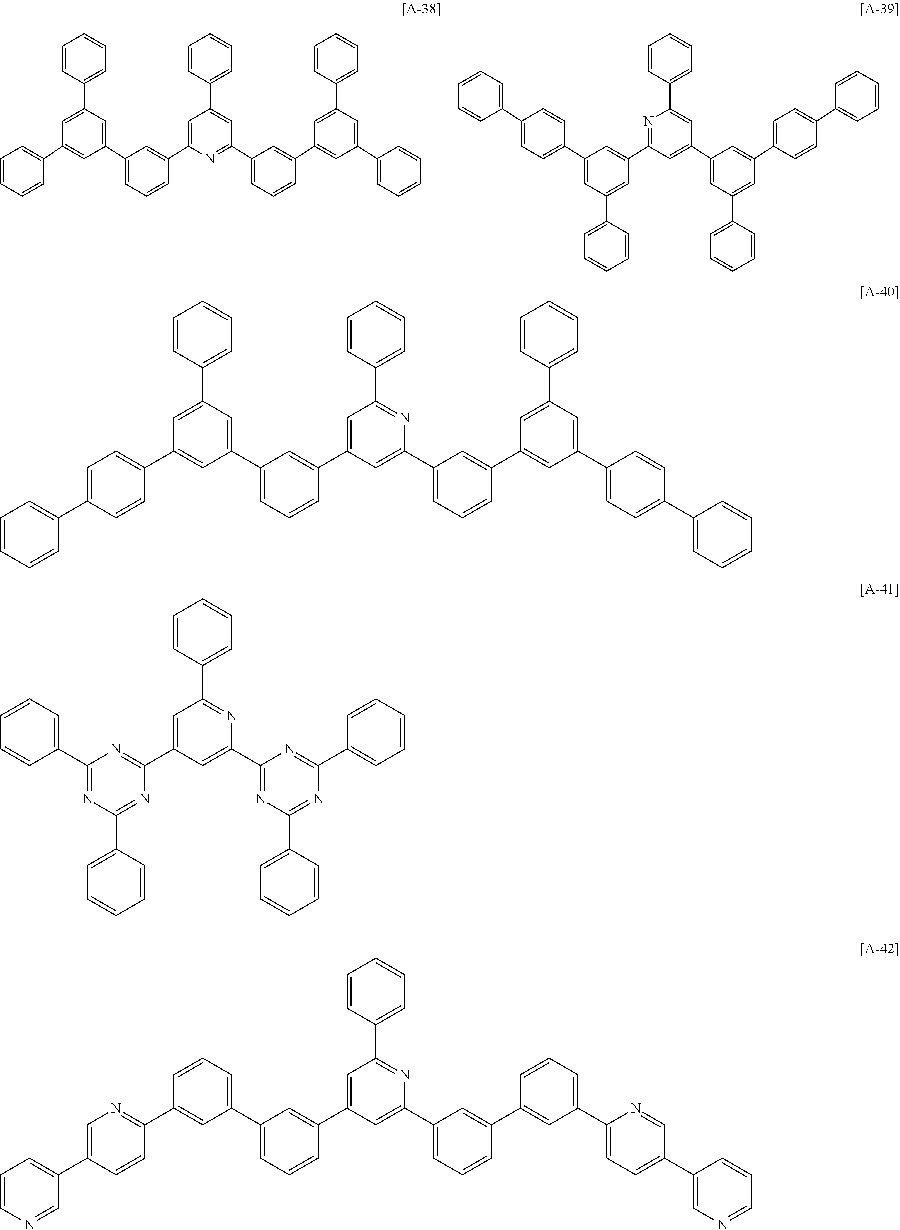

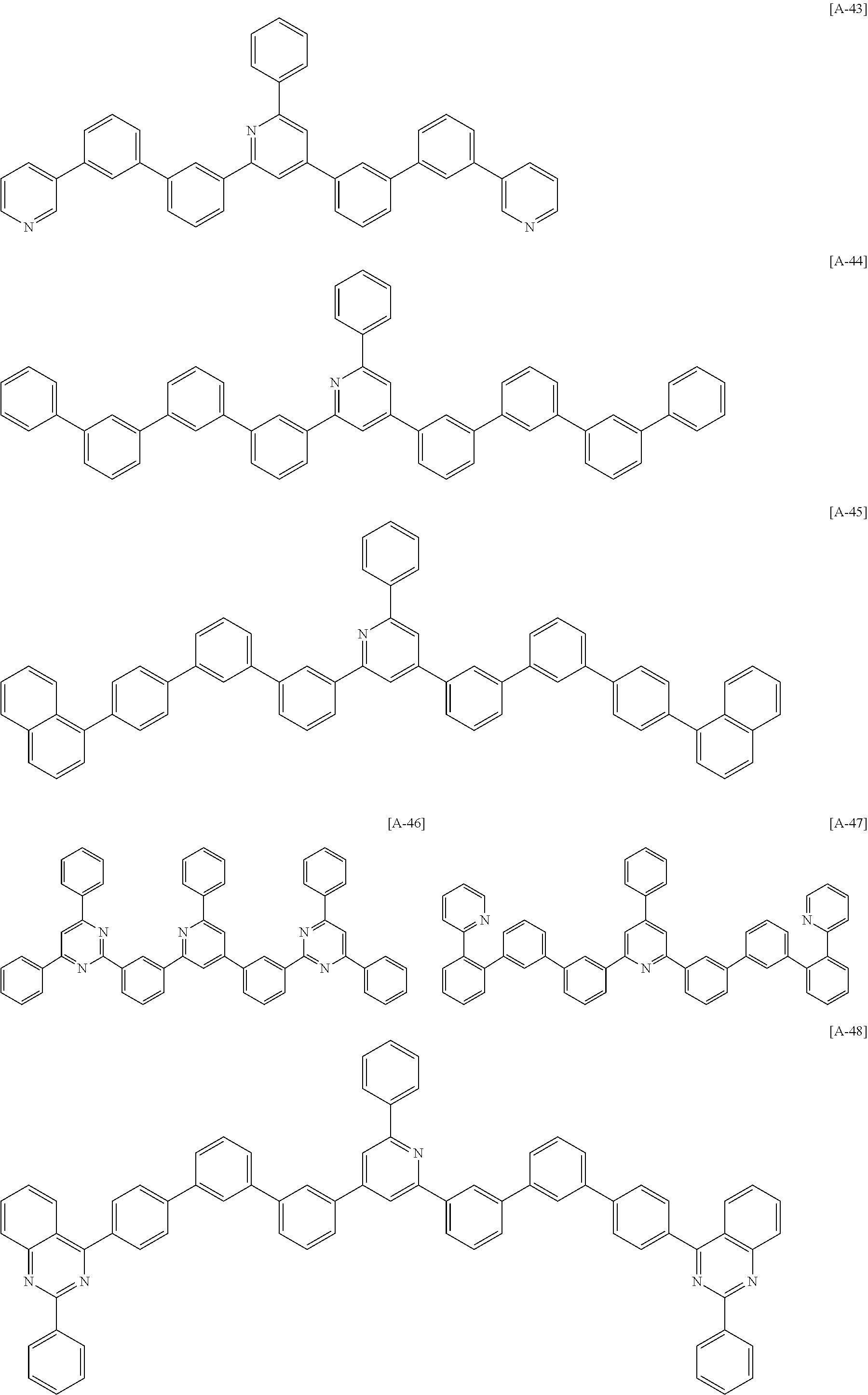

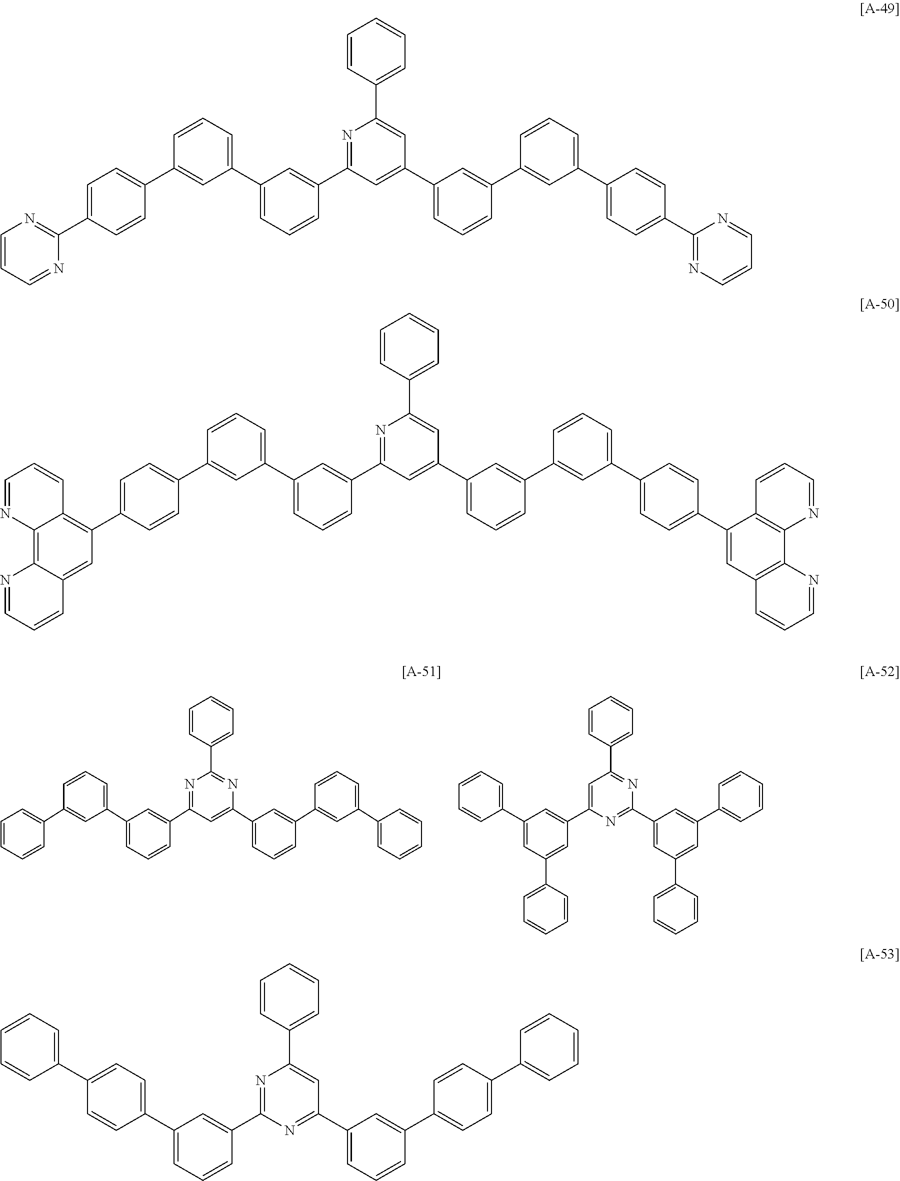

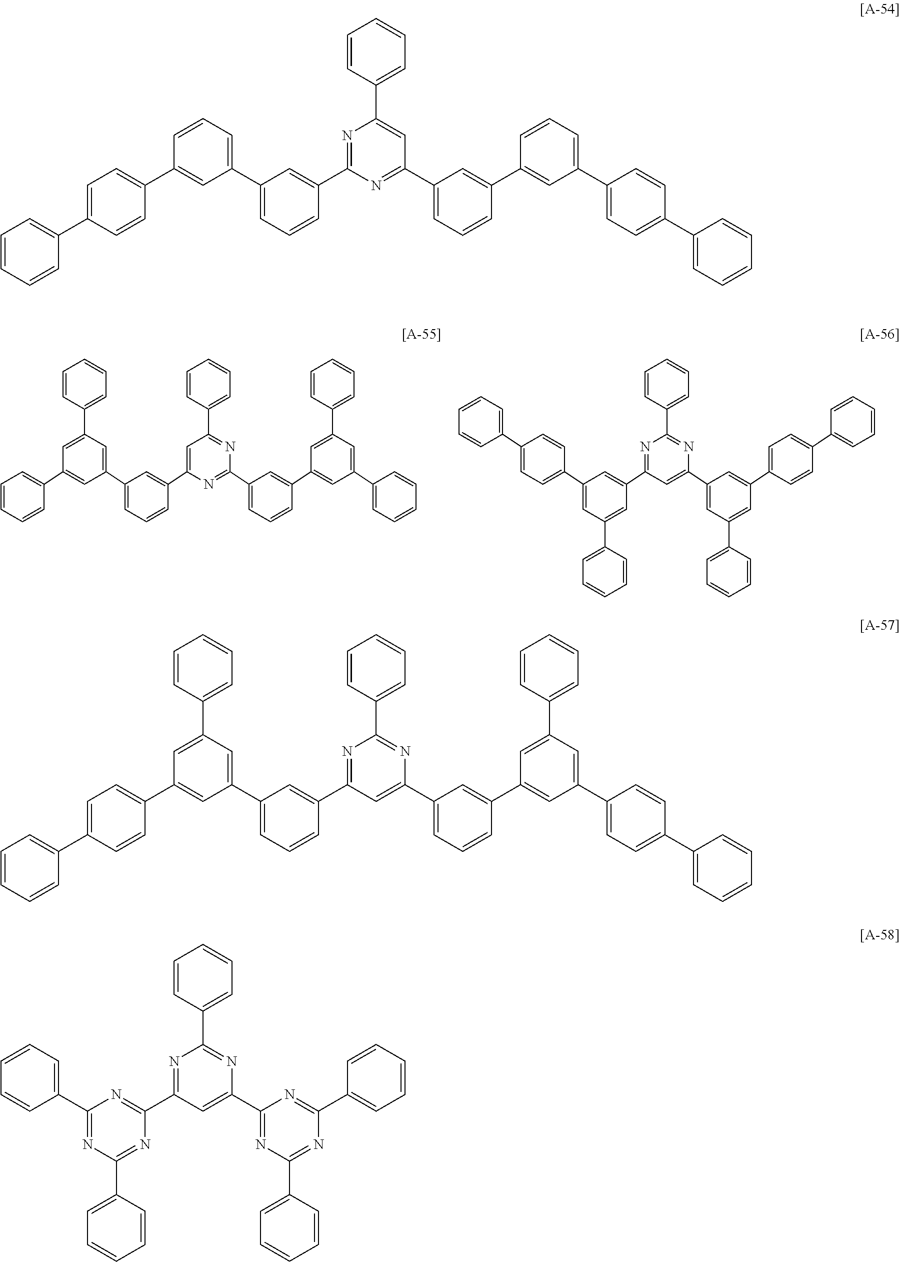

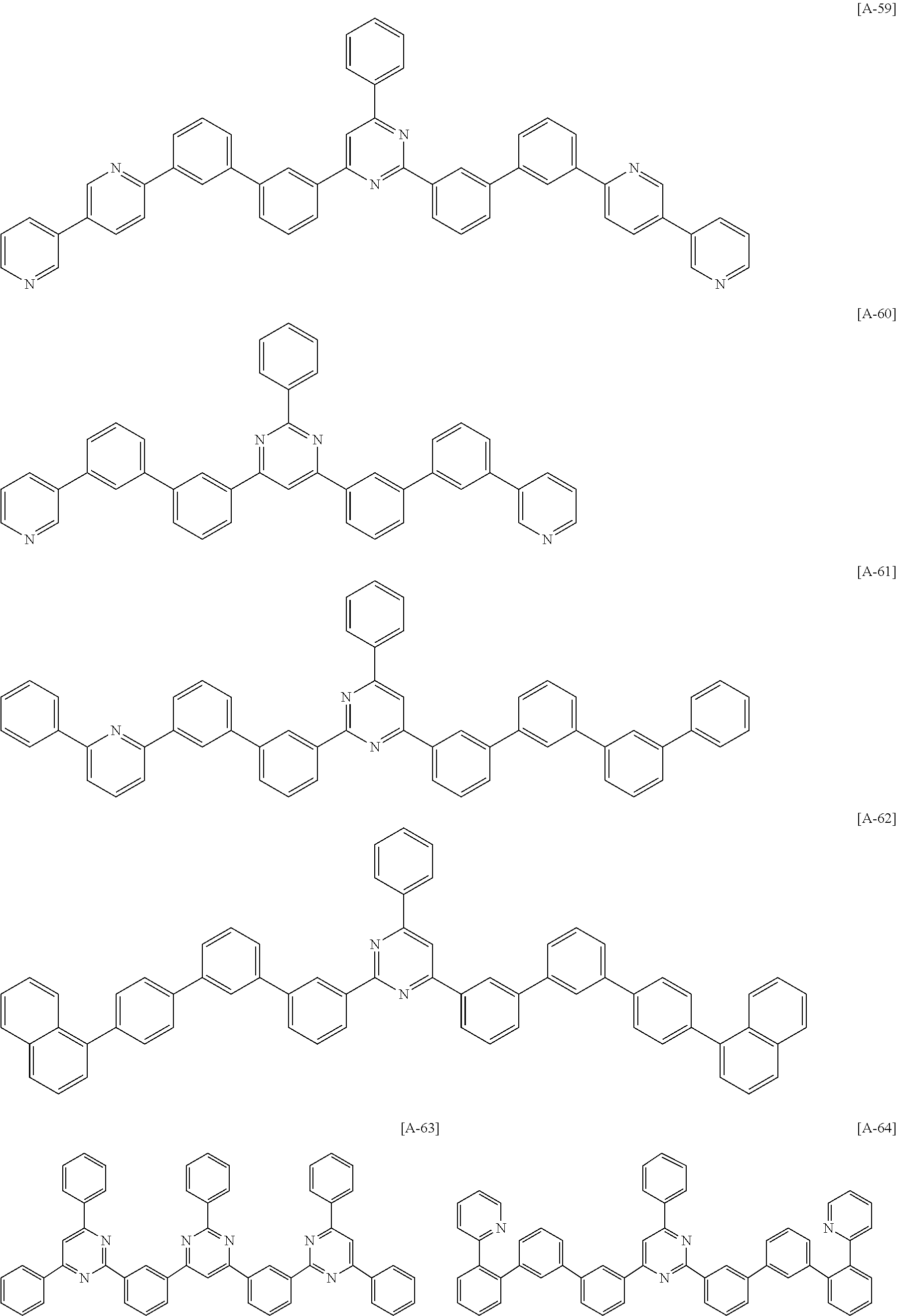

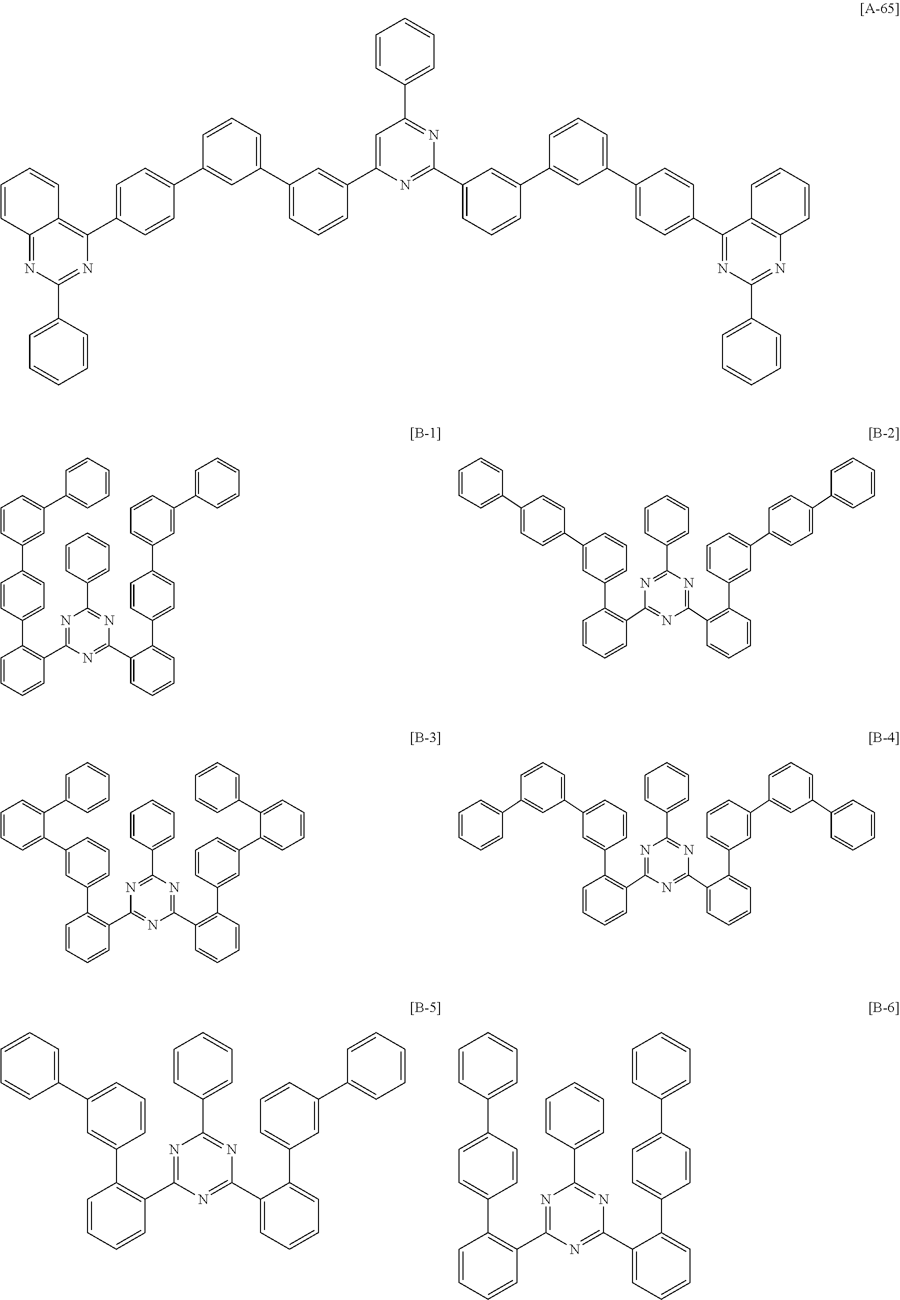

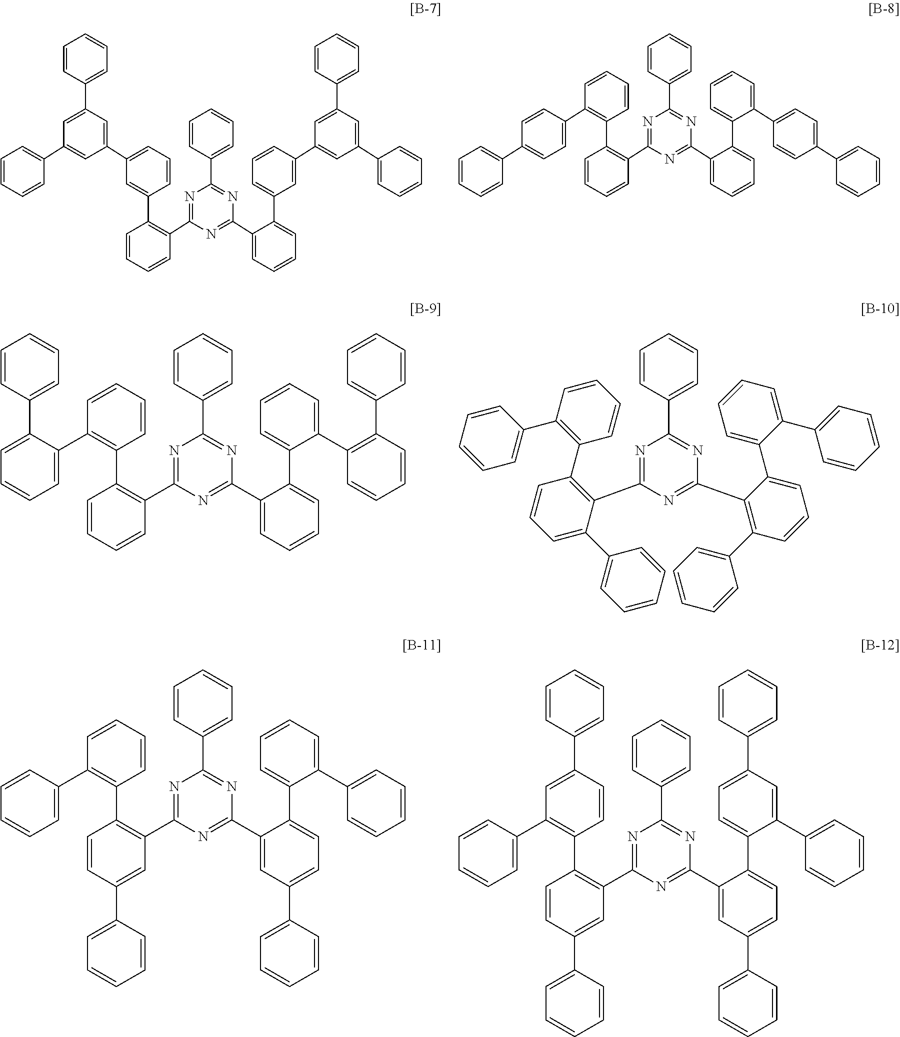

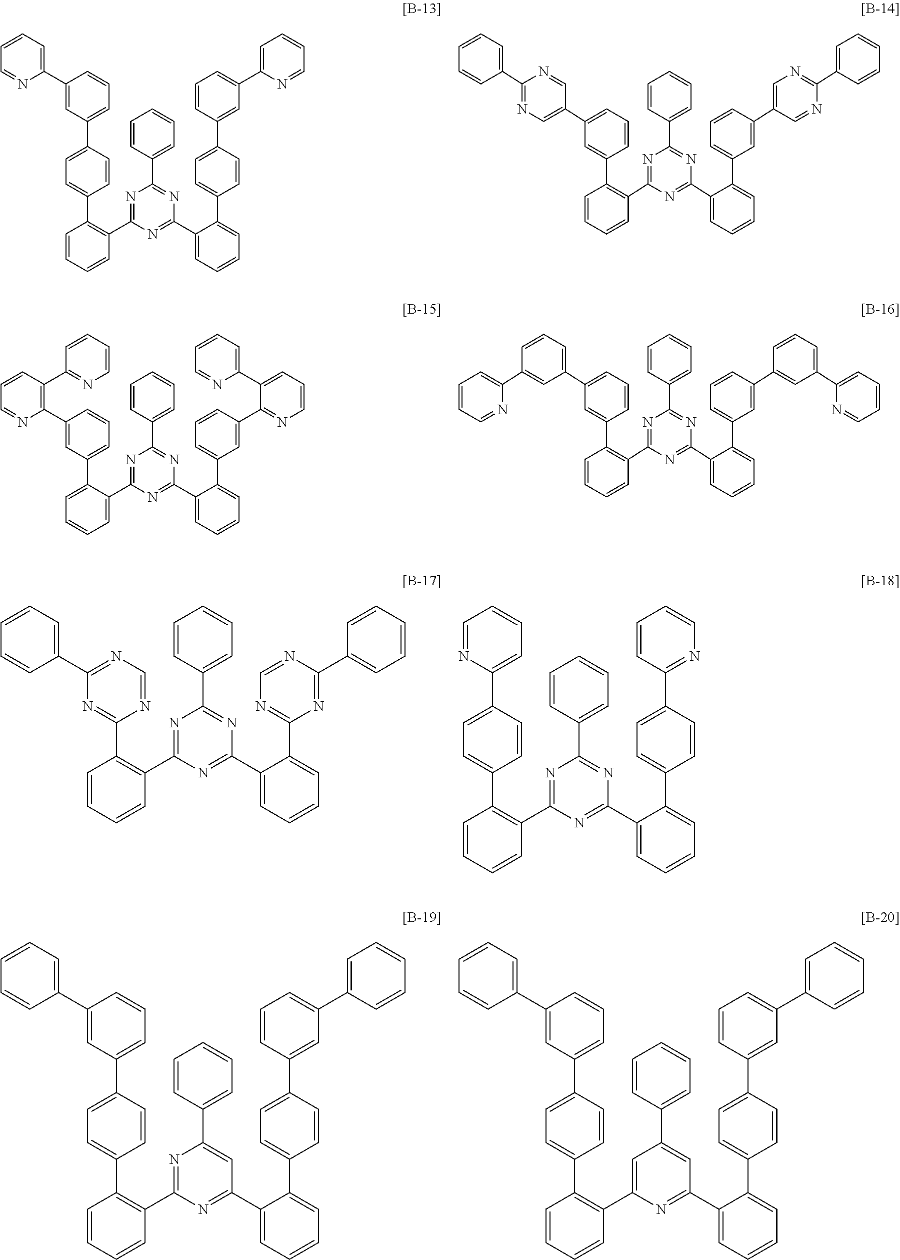

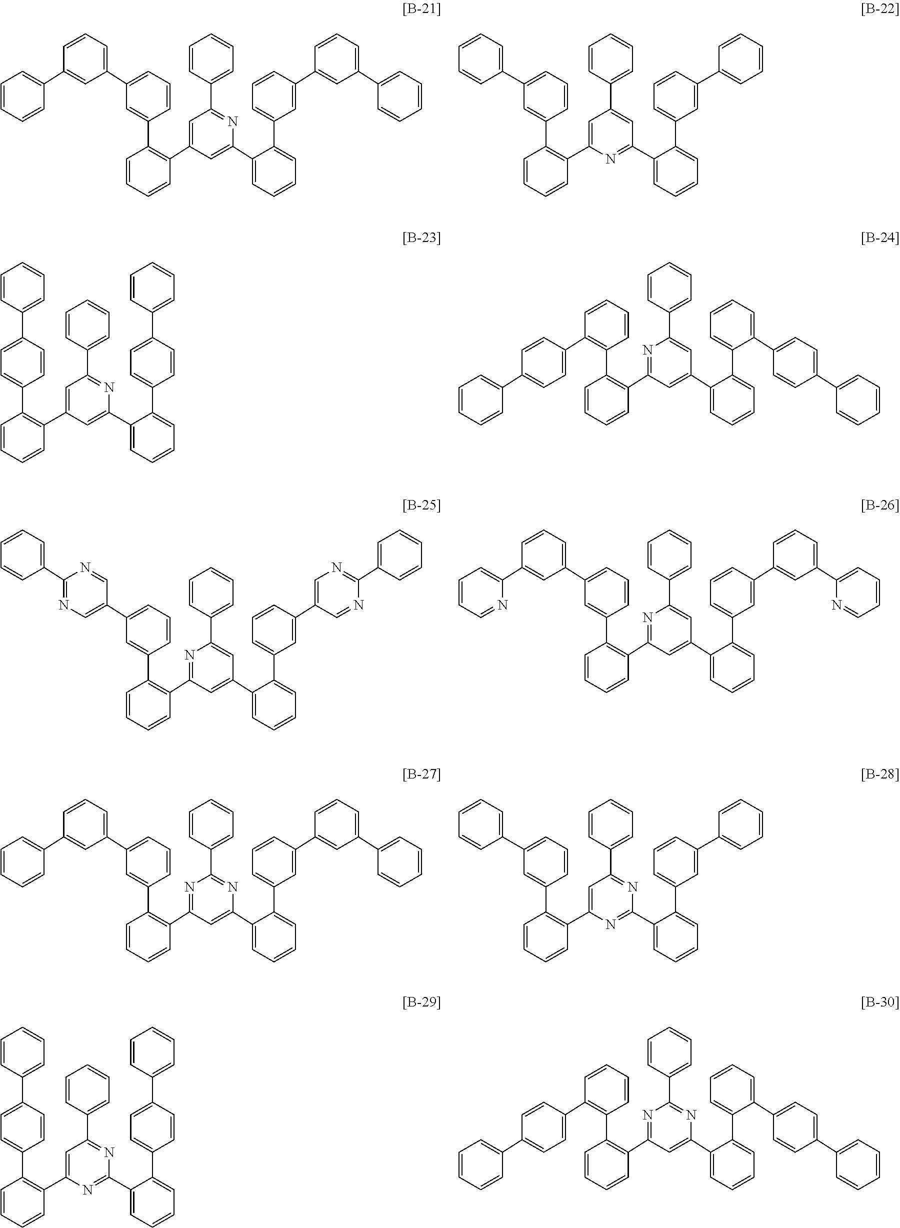

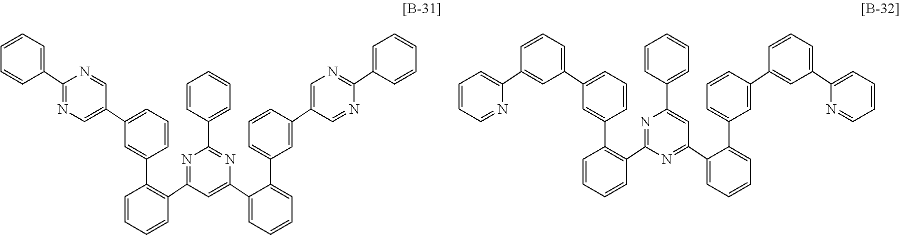 [SMILES: c1ccc(-c2ncc(-c3cccc(-c4ccccc4-c4cc(-c5ccccc5-c5cccc(-c6cnc(-c7ccccc7)nc6)c5)nc(-c5ccccc5)n4)c3)cn2)cc1 ; c1ccc(-c2cc(-c3ccccc3-c3cccc(-c4cccc(-c5ccccn5)c4)c3)nc(-c3ccccc3-c3cccc(-c4cccc(-c5ccccn5)c4)c3)n2)cc1]

The compounds may be used for an organic optoelectric device.

Hereinafter, an organic optoelectric device including the compound is described.

In another embodiment of the present invention, an organic optoelectric device includes an anode and a cathode facing each other and at least one organic layer between the anode and the cathode, wherein the organic layer includes the compound.

The organic layer may include an emission layer, and the emission layer may include the compound of the present invention.

Specifically, the compound may be included as a host of the emission layer.

In one embodiment of the present invention, the organic layer may include at least one auxiliary layer selected from a hole injection layer (HIL), a hole transport layer (HTL), a hole transport auxiliary layer, an electron transport auxiliary layer, an electron transport layer (ETL), and an electron injection layer (EIL), and the auxiliary layer includes the compound.

The organic optoelectric device may be any device to convert electrical energy into photoenergy and vice versa without particular limitation, and may be, for example an organic photoelectric device, an organic light emitting diode, an organic solar cell, and an organic photo-conductor drum.

Herein, an organic light emitting diode as one example of an organic optoelectric device is described referring to drawings.

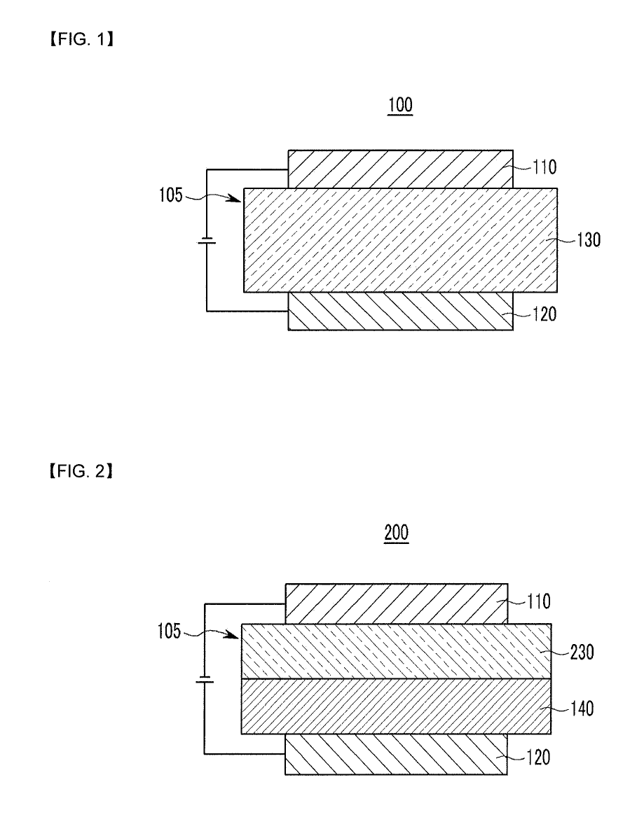

FIGS. 1 and 2 are cross-sectional views of each organic light emitting diode according to one embodiment.

Referring to FIG. 1, an organic light emitting diode 100 according to one embodiment includes an anode 120 and a cathode 110 facing each other and an organic layer 105 interposed between the anode 120 and cathode 110.

The anode 120 may be made of a conductor having a large work function to help hole injection, and may be for example metal, metal oxide and/or a conductive polymer. The anode 120 may be, for example a metal such as nickel, platinum, vanadium, chromium, copper, zinc, and gold or an alloy thereof; metal oxide such as zinc oxide, indium oxide, indium tin oxide (ITO), indium zinc oxide (IZO), and the like; a combination of metal and oxide such as ZnO and Al or SnO₂ and Sb; a conductive polymer such as poly(3-methylthiophene), poly(3,4-(ethylene-1,2-dioxy)thiophene) (PEDT), polypyrrole, and polyaniline, but is not limited thereto.

The cathode 110 may be made of a conductor having a small work function to help electron injection, and may be for example metal, metal oxide and/or a conductive polymer. The cathode 110 may be for example a metal or an alloy thereof such as magnesium, calcium, sodium, potassium, titanium, indium, yttrium, lithium, gadolinium, aluminum silver, tin, lead, cesium, barium, and the like; a multi-layer structure material such as LiF/Al, LiO₂/Al, LiF/Ca, LiF/Al and BaF₂/Ca, but is not limited thereto.

The organic layer 105 includes an emission layer 130 including the compound.

The emission layer 130 may include, for example the organic compound at alone, or a mixture of at least two kinds and may include another compound different from the compound. When the compound is mixed with another compound, they may be, for example a host and a dopant, and the compound may be, for example a host. The host may be, for example a phosphorescent host or fluorescent host, and may be, for example a phosphorescent host.

When the compound is a host, the dopant may be an inorganic, organic, or organic/inorganic compound, and may be selected from known dopants.

Referring to FIG. 2, an organic light emitting diode 200 further includes a hole auxiliary layer 140 in addition to an emission layer 230. The hole auxiliary layer 140 may improve hole injection and/or hole mobility between the anode 120 and the emission layer 230 and may block electrons. The hole auxiliary layer 140 may include, for example at least one of a hole transport layer, a hole injection layer and/or an electron blocking layer. The compound may be included in the hole auxiliary layer 140.

Even not shown in FIG. 1 or 2, the organic layer 105 may further include an electron injection layer, an electron transport layer, an auxiliary electron transport layer, a hole transport layer, an auxiliary hole transport layer, a hole injection layer or a combination thereof. The compound of the present invention may be included in the organic layer. The organic light emitting diodes 100 and 200 may be manufactured by forming an anode or a cathode on a substrate, forming an organic layer in accordance with a dry coating method such as evaporation, sputtering, plasma plating, and ion plating or a wet coating method such as spin coating, dipping, and flow coating; and forming a cathode or an anode thereon.

The organic light emitting diode may be applied to an organic light emitting diode (OLED) display.

### MODE FOR INVENTION

Hereinafter, the embodiments are illustrated in more detail with reference to examples. These examples, however, are not in any sense to be interpreted as limiting the scope of the invention.

### (Preparation of Compound)

A compound was synthesized through the following steps as specific examples of a compound according to the present invention.

### Synthesis Example 1: Synthesis of Intermediate L-1

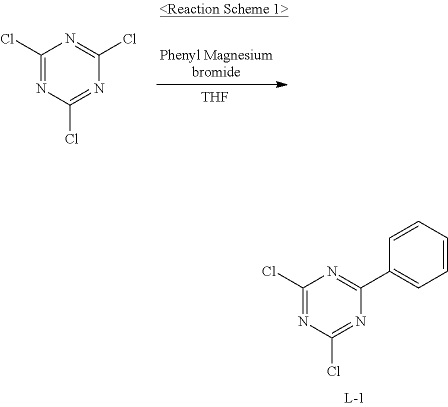 [SMILES: Clc1nc(Cl)nc(Cl)n1 ; Clc1nc(Cl)nc(-c2ccccc2)n1 ; "THF"]

30 g (162.68 mmol) of 2,4,6-trichloro-1,3,5-triazine was put in a 500 mL flask and dissolved in 325 ml of a tetrahydrofuran solvent. After cooling down the solvent with ice water, 54.23 ml (162.68 mmol) of phenylmagnesium bromide having a concentration of 3 M were slowly dropped thereto through a dropping funnel under a nitrogen stream. When the phenylmagnesium bromide was completely added thereto, the mixture was agitated for 30 minutes, and then, water was added thereto, completing a reaction. The water was separated from the tetrahydrofuran and removed, and then, the tetrahydrofuran was removed through distiller, obtaining a solid. The solid was agitated with 100 ml of methanol and then, filtered. Then, the solid was agitated with 100 ml of hexane again and then, filtered, obtaining an intermediate L-1 (27 g, 73% of a yield).

calcd. C9H5Cl2N3: C, 47.82; H, 2.23; C1, 31.37; N, 18.59; found: C, 47.56; H, 2.12; C1, 31.42; N, 18.43;

### Synthesis Examples 2 and 3: Synthesis of Intermediates L-2 and L-3

Intermediates L-2 and L-3 as specific examples of a compound according to the present invention were synthesized according to the following Reaction Schemes 2 and 3 in the same method as the L-1 of Synthesis Example 1.

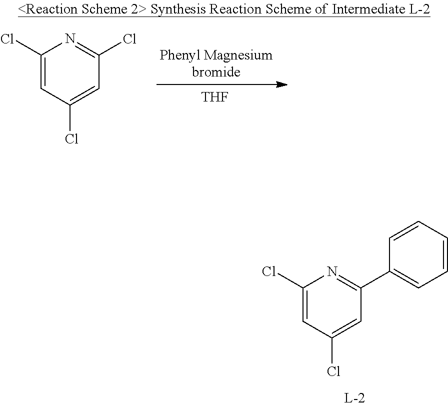 [SMILES: Clc1cc(Cl)nc(Cl)c1 ; Clc1cc(Cl)nc(-c2ccccc2)c1 ; "THF"]

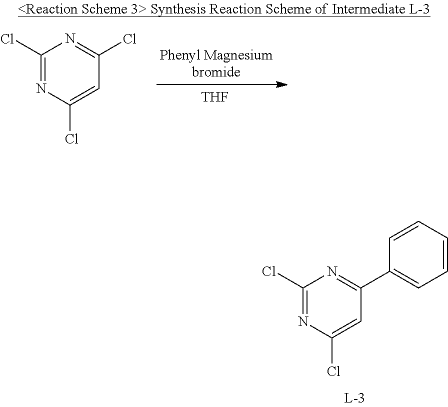 [SMILES: Clc1cc(Cl)nc(Cl)n1 ; Clc1cc(-c2ccccc2)nc(Cl)n1 ; "THF"]

### Synthesis Process of Intermediates L-4, L-5 and L-6

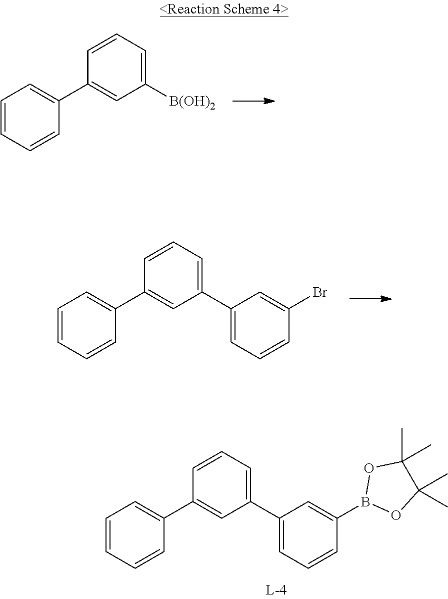 [SMILES: OB(O)c1cccc(-c2ccccc2)c1 ; Brc1cccc(-c2cccc(-c3ccccc3)c2)c1 ; CC1(C)OB(c2cccc(-c3cccc(-c4ccccc4)c3)c2)OC1(C)C]

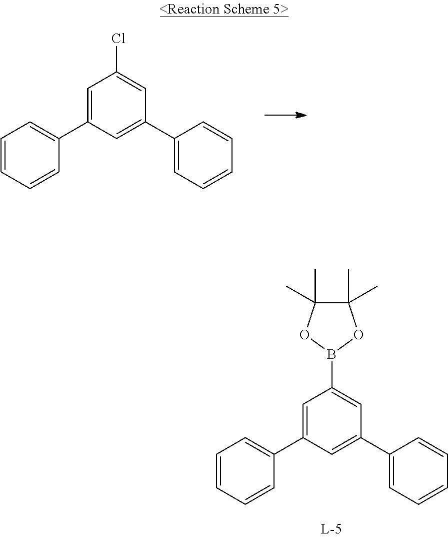 [SMILES: Clc1cc(-c2ccccc2)cc(-c2ccccc2)c1 ; CC1(C)OB(c2cc(-c3ccccc3)cc(-c3ccccc3)c2)OC1(C)C]

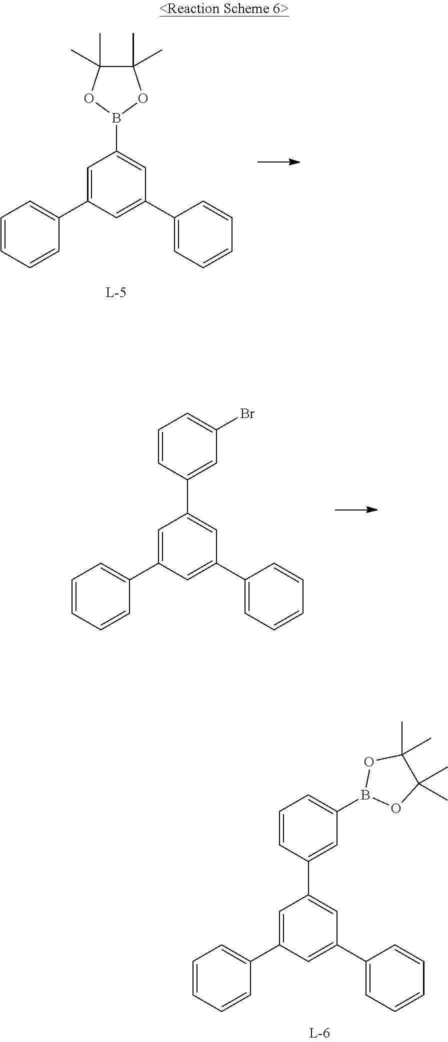 [SMILES: CC1(C)OB(c2cc(-c3ccccc3)cc(-c3ccccc3)c2)OC1(C)C ; Brc1cccc(-c2cc(-c3ccccc3)cc(-c3ccccc3)c2)c1 ; CC1(C)OB(c2cccc(-c3cc(-c4ccccc4)cc(-c4ccccc4)c3)c2)OC1(C)C]

### Synthesis Example 4: Synthesis of Compound, 3-Bromo-1,1′:3,1″-Terphenyl

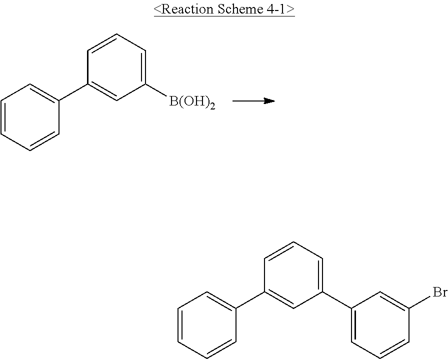 [SMILES: OB(O)c1cccc(-c2ccccc2)c1 ; Brc1cccc(-c2cccc(-c3ccccc3)c2)c1]

50.0 g (252.49 mmol) of the intermediate, [1,1′-biphenyl]-3-yl boronic acid, 92.86 g (328.23 mmol) of 1-bromo-3-iodobenzene, 69.79 g (504.97 mmol) of potassium carbonate, and 14.59 g (12.62 mmol) of Pd(PPh₃)₄ (tetrakis(triphenyl phosphine)palladium (0)) were added to 500 mL of tetrahydrofuran and 250 mL of water in a 2000 ml flask, and the mixture was heated and refluxed for 10 hours under a nitrogen stream. Then, a solid crystallized by adding 1500 mL of methanol to the obtained mixture was filtered, dissolved in dichloromethane and then, filtered with silica gel/Celite and then, recrystallized with methanol after removing the organic solvent in an appropriate amount, obtaining a compound A-1 (55.32 g, 71% of a yield). The element analysis result of the compound, 3-bromo-1,1′:3,1″-terphenyl was provided as follows.

calcd. C₁₈H₁₃Br: C, 69.92; H, 4.24; Br, 25.84; found: C, 69.62; H, 4.11; Br, 25.75;

### Synthesis Example 5: Synthesis of Intermediate L-4

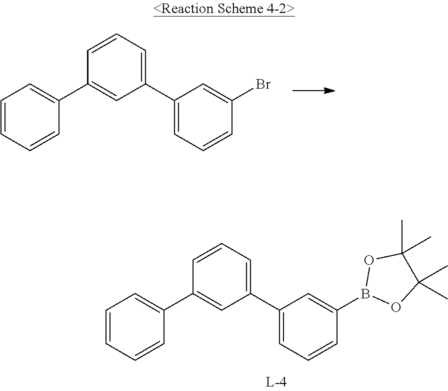 [SMILES: Brc1cccc(-c2cccc(-c3ccccc3)c2)c1 ; CC1(C)OB(c2cccc(-c3cccc(-c4ccccc4)c3)c2)OC1(C)C]

50.0 g (161.71 mmol) of the intermediate, 3-bromo-1,1′: 3,1″-terphenyl, 53.38 g (210.22 mmol) of bispinacolato diboron, 47.61 g (485.12 mmol) of potassium acetate, and 14.59 g (12.62 mmol) of Pd(dppf)Cl₂ were added to 580 mL of toluene in a 1000 ml flask, and the mixture was heated and refluxed for 10 hours under a nitrogen stream. Then, a solid crystallized by adding 1500 mL of methanol to the obtained mixture was filtered, dissolved in dichloromethane, filtered with silica gel/Celite and then, recrystallized with hexane after removing the organic solvent in an appropriate amount, obtaining a compound L-4 (45.6 g, 79% of a yield). The elemental analysis result of the compound L-4 was provided as follows.

calcd. C₂₄H₂₅BO₂: C, 80.91; H, 7.07; B, 3.03; O, 8.98; found: C, 80.87; H, 7.13; B, 3.24; O, 8.76;

### Synthesis Example 6: Synthesis of Intermediate L-5

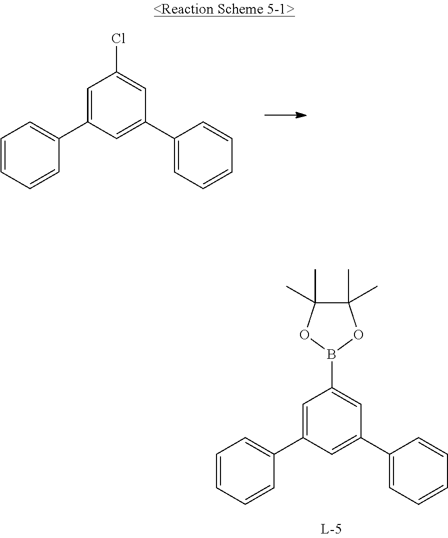 [SMILES: Clc1cc(-c2ccccc2)cc(-c2ccccc2)c1 ; CC1(C)OB(c2cc(-c3ccccc3)cc(-c3ccccc3)c2)OC1(C)C]

50.0 g (188.86 mmol) of the intermediate, 5-chloro-1,1′: 3,1″-terphenyl, 62.35 g (245.51 mmol) of bispinacolato diboron, 55.60 g (566.67 mmol) of potassium acetate, and 9.25 g (11.33 mmol) of Pd(dppf)Cl₂ were added to 670 mL of dimethyl formamide in a 1000 ml flask, and the mixture was heated and refluxed for 10 hours under a nitrogen stream. Then, a solid crystallized by adding 1500 mL of methanol to the obtained mixture was filtered, dissolved in dichloromethane, filtered with silica gel/Celite and then, recrystallized with hexane after removing the organic solvent in an appropriate amount, obtaining a compound L-5 (46.34 g, 69% of a yield). The element analysis result of the compound L-5 was provided as follows.

calcd. C₂₄H₂₅BO₂: C, 80.91; H, 7.07; B, 3.03; O, 8.98; found: C, 80.34; H, 7.53; B, 3.21; O, 8.64;

### Synthesis Example 7: Synthesis of Compound, 3-bromo-5′-phenyl-1,1′: 3,1″-terphenyl

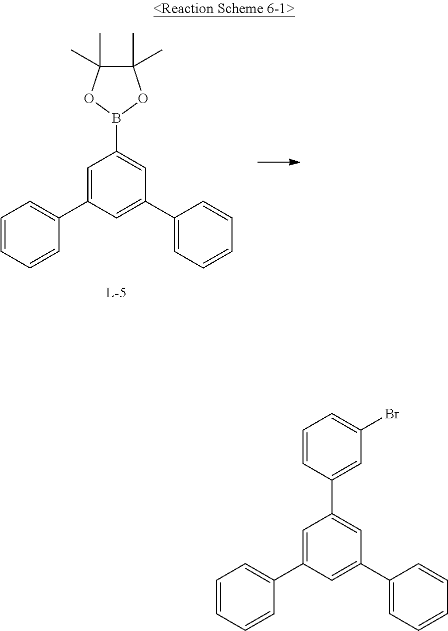 [SMILES: CC1(C)OB(c2cc(-c3ccccc3)cc(-c3ccccc3)c2)OC1(C)C ; Brc1cccc(-c2cc(-c3ccccc3)cc(-c3ccccc3)c2)c1]

70.0 g (196.48 mmol) of the intermediate L-5, 72.26 g (255.42 mmol) of 1-bromo-3-iodobenzene, 54.31 g (392.96 mmol) of potassium carbonate, and 11.35 g (9.82 mmol) of Pd(PPh₃)₄(Tetrakis(triphenylphosphine)palladium (0)) were added to 400 mL of tetrahydrofuran and 200 mL of water in a 2000 ml flask, and the mixture was heated and refluxed for 10 hours under a nitrogen stream. Then, a solid crystallized by adding 1500 mL of methanol to the obtained mixture was filtered, dissolved in dichloromethane, filtered with silica gel/Celite and then, recrystallized with methanol by removing the organic solvent in an appropriate amount, obtaining a compound, 3-bromo-5′-phenyl-1,1′: 3,1″-terphenyl (58.63 g, 77% of a yield). The element analysis result of the compound 3-bromo-5′-phenyl-1,1′: 3,1″-terphenyl was provided as follows.

calcd. C₂₄H₁₇Br: C, 74.81; H, 4.45; Br, 20.74; found: C, 74.65; H, 4.35; Br, 20.87;

### Synthesis Example 8: Synthesis of Intermediate L-6

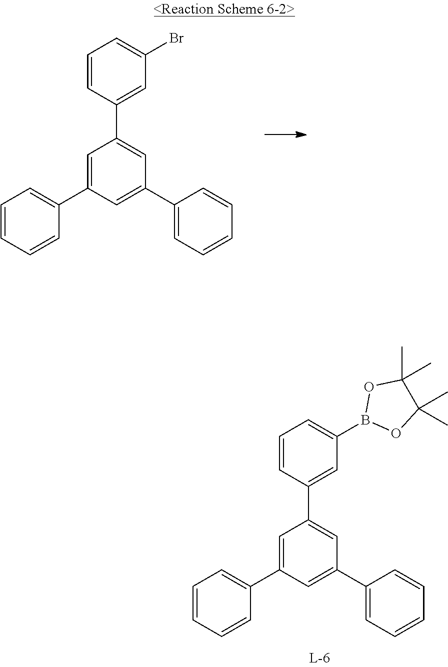 [SMILES: Brc1cccc(-c2cc(-c3ccccc3)cc(-c3ccccc3)c2)c1 ; CC1(C)OB(c2cccc(-c3cc(-c4ccccc4)cc(-c4ccccc4)c3)c2)OC1(C)C]

50.0 g (139.55 mmol) of the intermediate, 3-bromo-5′-phenyl-1,1′: 3,1″-terphenyl, 46.07 g (181.41 mmol) of bispinacolato diboron, 41.09 g (418.64 mmol) of potassium acetate, and 6.84 g (8.37 mmol) of Pd(dppf)Cl₂ were added to 500 mL of toluene in a 1000 ml flask, and the mixture was heated and refluxed for 10 hours under a nitrogen stream. Then, a solid crystallized by adding 1500 mL of methanol to the obtained mixture was filtered, dissolved in dichloromethane, filtered with silica gel/Celite and then, recrystallized with hexane after removing the organic solvent in an appropriate amount, obtaining a compound L-6 (51.23 g, 85% of a yield). The element analysis result of the compound L-6 was provided as follows.

calcd. C₂₄H₂₅BO₂: C, 80.91; H, 7.07; B, 3.03; O, 8.98; found: C, 80.87; H, 7.13; B, 3.24; O, 8.76;

### Synthesis of Intermediates L-7, L-8, L-9, L-10

Intermediates L-7, L-8, L-9 and L-10 as specific examples of a compound of the present invention were synthesized according to the same method as the intermediates L-4, L-5, and L-6 according to Synthesis Examples 4 to 8 (three basic reactions: a Suzuki reaction, a Br boration reaction, a boration reaction of CI)

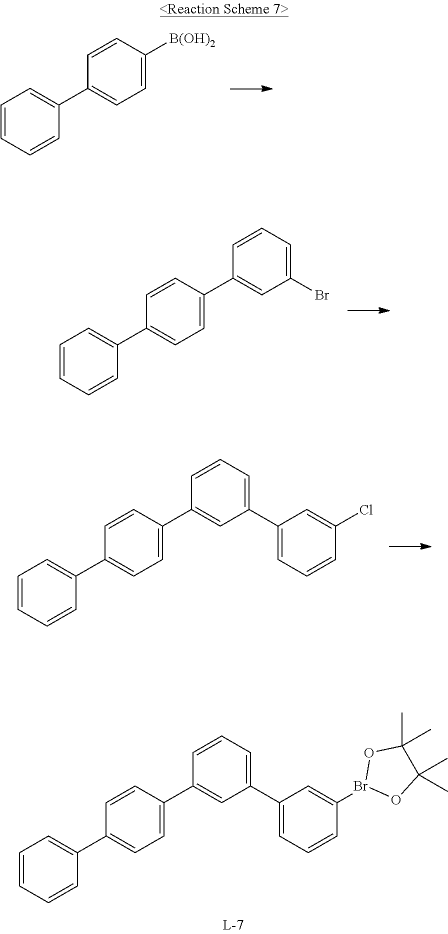

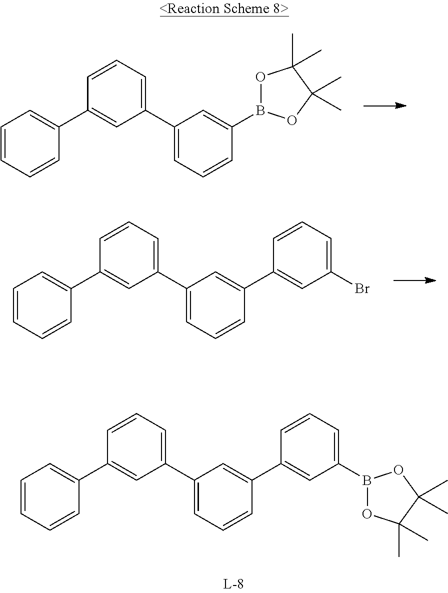 [SMILES: CC1(C)OB(c2cccc(-c3cccc(-c4ccccc4)c3)c2)OC1(C)C ; Brc1cccc(-c2cccc(-c3cccc(-c4ccccc4)c3)c2)c1 ; CC1(C)OB(c2cccc(-c3cccc(-c4cccc(-c5ccccc5)c4)c3)c2)OC1(C)C]

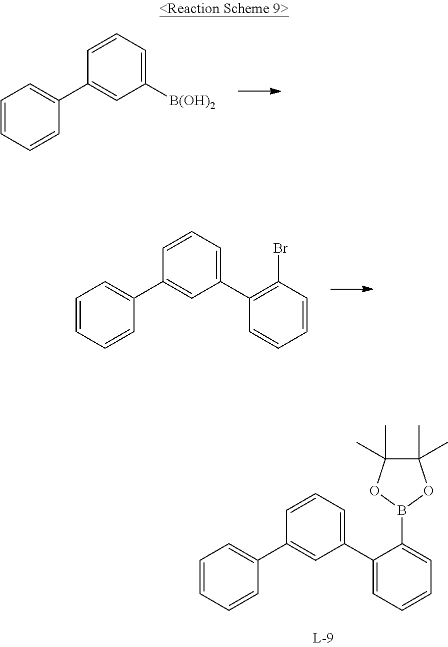 [SMILES: OB(O)c1cccc(-c2ccccc2)c1 ; Brc1ccccc1-c1cccc(-c2ccccc2)c1 ; CC1(C)OB(c2ccccc2-c2cccc(-c3ccccc3)c2)OC1(C)C]

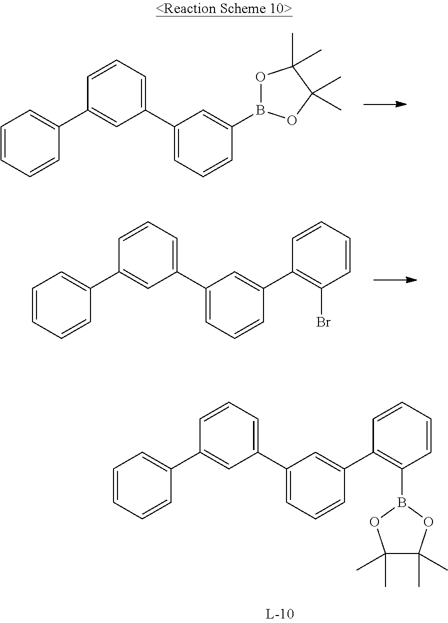 [SMILES: CC1(C)OB(c2cccc(-c3cccc(-c4ccccc4)c3)c2)OC1(C)C ; Brc1ccccc1-c1cccc(-c2cccc(-c3ccccc3)c2)c1 ; CC1(C)OB(c2ccccc2-c2cccc(-c3cccc(-c4ccccc4)c3)c2)OC1(C)C]

### Synthesis Example 1: Synthesis of Compound A-1

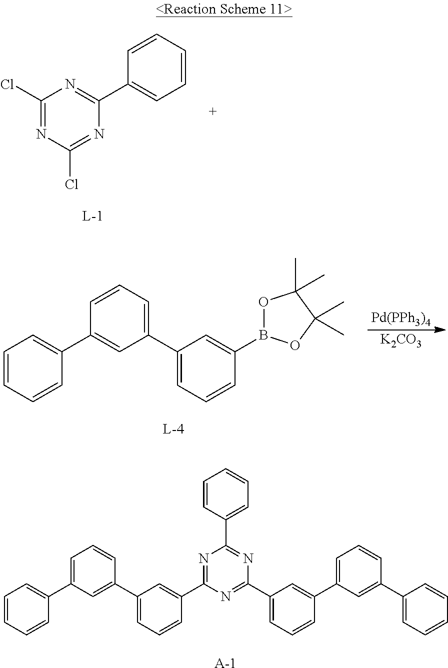

5.0 g (22.12 mmol) of the intermediate L-1 (2,4-dichloro-6-phenyl-s-triazine), 18.12 g (50.87 mmol) of the intermediate L-4, 7.64 g (55.30 mmol) of potassium carbonate, and 1.28 g (1.11 mmol) of Pd(PPh₃)₄ (Tetrakis(triphenyl phosphine)palladium (0)) were added to 100 mL of tetrahydrofuran and 30 mL of water in a 250 mL flask, and the mixture was heated and refluxed for 10 hours under a nitrogen stream. Then, a solid crystallized by adding 500 mL of methanol to the obtained mixture was filtered, dissolved in monochlorobenzene, filtered with silica gel/Celite and then, recrystallized with methanol after removing the organic solvent in an appropriate amount, obtaining a compound A-1 (11.3 g, 83% of a yield). The element analysis result of the compound A-1 is provided as follows.

calcd. C₄₅H₃₁N₃: C, 88.06; H, 5.09; N, 6.85; found: C, 87.94; H, 5.12; N, 6.76;

### Synthesis Example 2: Synthesis of Compound A-2

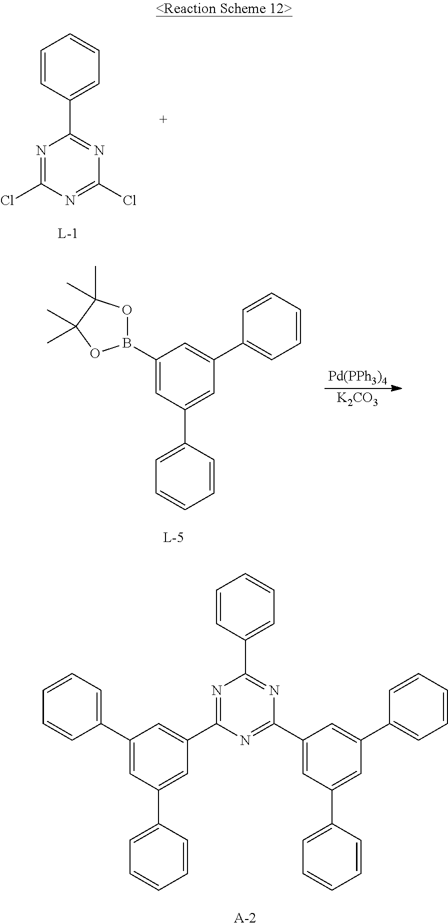

5.0 g (22.12 mmol) of the intermediate L-1 (2,4-dichloro-6-phenyl-s-triazine), 18.12 g (50.87 mmol) of the intermediate L-5, 7.64 g (55.30 mmol) of potassium carbonate, and 1.28 g (1.11 mmol) of Pd(PPh₃)₄ (Tetrakis(triphenyl phosphine)palladium (0)) were added to 100 mL of tetrahydrofuran and 30 mL of water in a 250 mL flask, and the mixture was heated and refluxed for 10 hours under a nitrogen stream. Then, a solid crystallized by adding 500 mL of methanol to the obtained mixture was filtered, dissolved in monochlorobenzene, filtered again with silica gel/Celite, and then, recrystallized with methanol after removing the organic solvent in an appropriate amount, obtaining a compound A-2 (8.5 g, 63% of a yield). The element analysis result of the compound A-2 was provided as follows.

calcd. C₄₅H₃₁N₃: C, 88.06; H, 5.09; N, 6.85; found: C, 88.16; H, 5.23; N, 6.63;

### Synthesis Example 3: Synthesis of Compound A-5

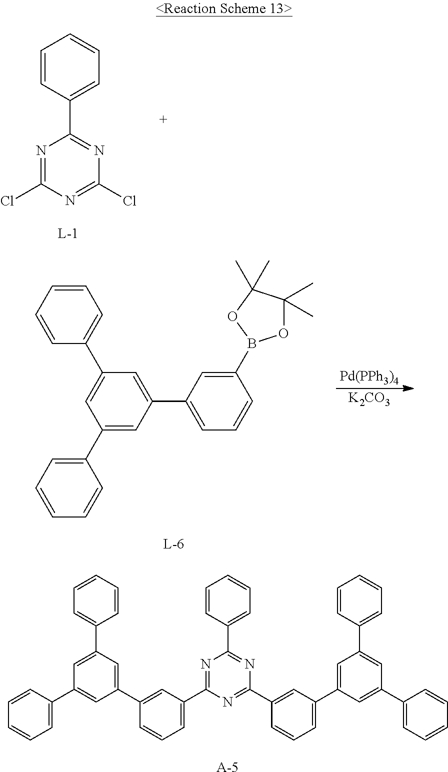 [SMILES: Clc1nc(Cl)nc(-c2ccccc2)n1 ; CC1(C)OB(c2cccc(-c3cc(-c4ccccc4)cc(-c4ccccc4)c3)c2)OC1(C)C ; "K2CO3" ; c1ccc(-c2cc(-c3ccccc3)cc(-c3cccc(-c4nc(-c5ccccc5)nc(-c5cccc(-c6cc(-c7ccccc7)cc(-c7ccccc7)c6)c5)n4)c3)c2)cc1 ; "A-5" ; "Pd(PPh3)4"]

5.0 g (22.12 mmol) of the intermediate L-1 (2,4-dichloro-6-phenyl-s-triazine), 21.99 g (46.60 mmol) of the intermediate L-6, 7.64 g (55.30 mmol) of potassium carbonate, and 1.28 g (1.11 mmol) of Pd(PPh₃)₄ (tetrakis(triphenyl phosphine)palladium (0)) were added to 100 mL of tetrahydrofuran and 30 mL of water in a 250 mL flask, and the mixture was heated and refluxed for 10 hours under a nitrogen stream. Then, a solid crystallized by adding the obtained mixture to 500 mL of methanol was filtered, dissolved in monochlorobenzene, filtered again with silica gel/Celite, and recrystallized with methanol after removing the organic solvent in an appropriate amount, obtaining a compound A-5 (13.0 g, 77% of a yield). The element analysis result of the compound A-5 was provided as follows.

calcd. C₅₇H₃₉N₃: C, 89.38; H, 5.13; N, 5.49; found: C, 89.21; H, 5.04; N, 5.53;

### Synthesis Example 4: Synthesis of Compound A-7

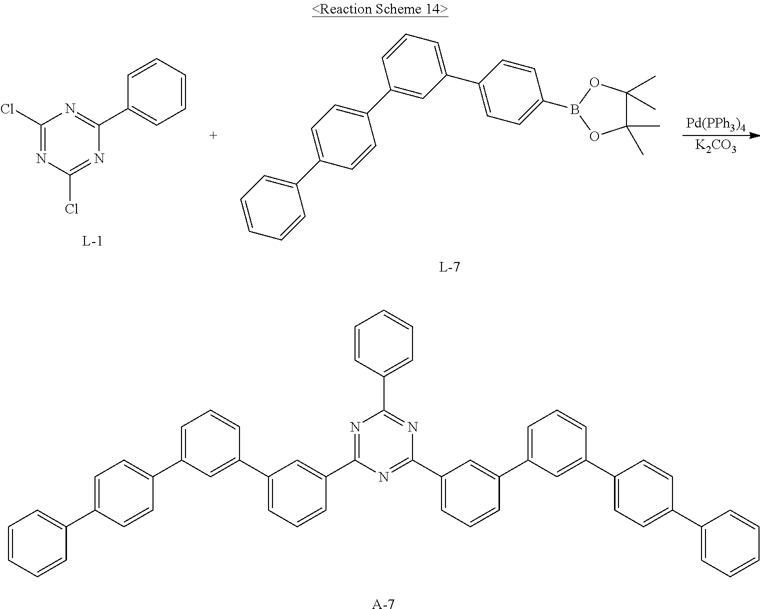

5.0 g (22.12 mmol) of the intermediate L-1 (2,4-dichloro-6-phenyl-s-triazine), 21.99 g (46.60 mmol) of the intermediate L-7, 7.64 g (55.30 mmol) of potassium carbonate, and 1.28 g (1.11 mmol) of Pd(PPh₃)₄ (Tetrakis(triphenyl phosphine)palladium (0)) were added to 100 mL of tetrahydrofuran and 30 mL of water in a 250 mL flask, and the mixture was heated and refluxed for 10 hours under a nitrogen stream. Then, a solid crystallized by adding 500 mL of methanol to the obtained mixture was filtered, dissolved in monochlorobenzene, filtered again with silica gel/Celite and then, recrystallized with methanol after removing the organic solvent in an appropriate amount, obtaining a compound A-7 (14.2 g, 84% of a yield). The element analysis result of the compound A-7 was provided as follows.

calcd. C₅₇H₃₉N₃: C, 89.38; H, 5.13; N, 5.49; found: C, 89.48; H, 5.33; N, 5.41;

### Synthesis Example 5: Synthesis of Compound A-25

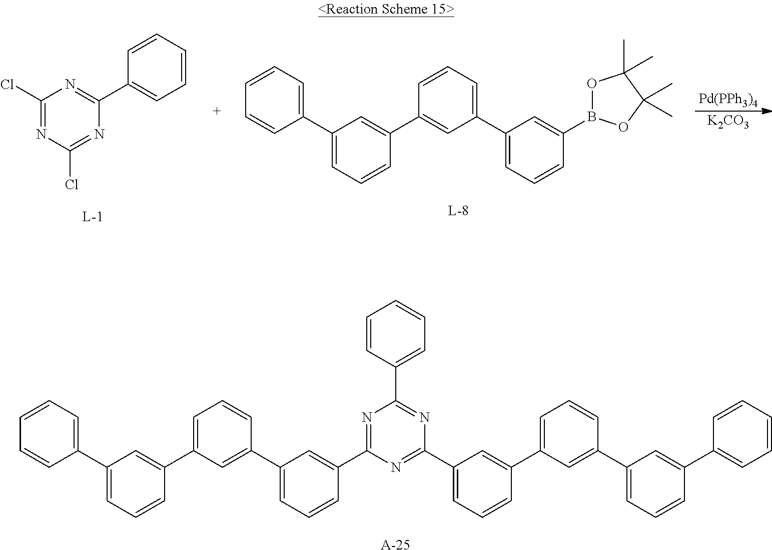

5.0 g (22.12 mmol) of the intermediate L-1 (2,4-dichloro-6-phenyl-s-triazine), 21.99 g (46.60 mmol) of the intermediate L-8, potassium carbonate 7.64 g (55.30 mmol), and 1.28 g (1.11 mmol) of Pd(PPh₃)₄ (Tetrakis(triphenyl phosphine)palladium (0)) were added to 100 mL of tetrahydrofuran and 30 mL of water in a 250 mL flask, and the mixture was heated and refluxed for 10 hours under a nitrogen stream. Then, a solid crystallized by adding the obtained mixture to 500 mL of methanol was filtered, dissolved in monochlorobenzene, filtered again with silica gel/Celite and then, recrystallized with methanol after removing the organic solvent in an appropriate amount, obtaining a compound A-25 (12.2 g, 72% of a yield). The element analysis result of the compound A-25 was provided as follows.

calcd. C₅₇H₃₉N₃: C, 89.38; H, 5.13; N, 5.49; found: C, 89.71; H, 5.46; N, 5.24;

### Synthesis Example 6: Synthesis of Compound B-5

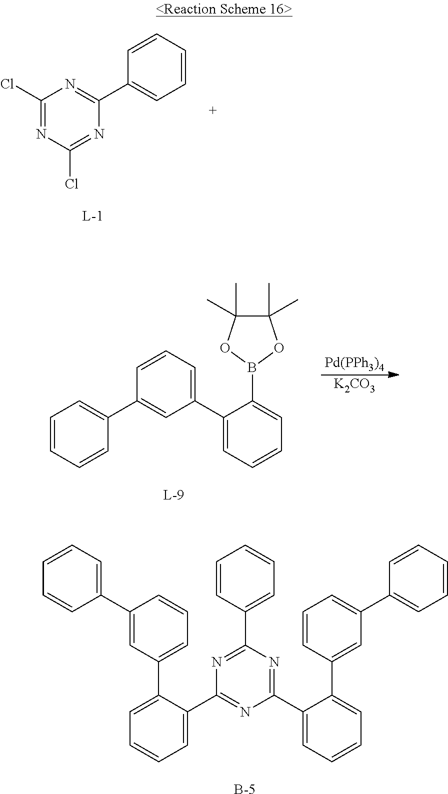

5.0 g (22.12 mmol) of the intermediate L-1 (2,4-dichloro-6-phenyl-s-triazine), 18.12 g (50.87 mmol) of the intermediate L-9, 7.64 g (55.30 mmol) of potassium carbonate, and 1.28 g (1.11 mmol) of Pd(PPh₃)₄ (Tetrakis(triphenyl phosphine)palladium (0)) were added to 100 mL of tetrahydrofuran and 30 mL of water in a 250 mL flask, and the mixture was heated and refluxed for 10 hours under a nitrogen stream. Then, a solid crystallized by adding the obtained mixture to 500 mL of methanol, dissolved in monochlorobenzene, filtered with silica gel/Celite and then, recrystallized with methanol after removing the organic solvent in an appropriate amount, obtaining a compound B-5 (10.3 g, 76% of a yield). The element analysis result of the compound B-5 was provided as follows.

calcd. C₄₅H₃₁N₃: C, 88.06; H, 5.09; N, 6.85; found: C, 87.84; H, 5.134; N, 6.75;

### Synthesis Example 7: Synthesis of Compound B-4

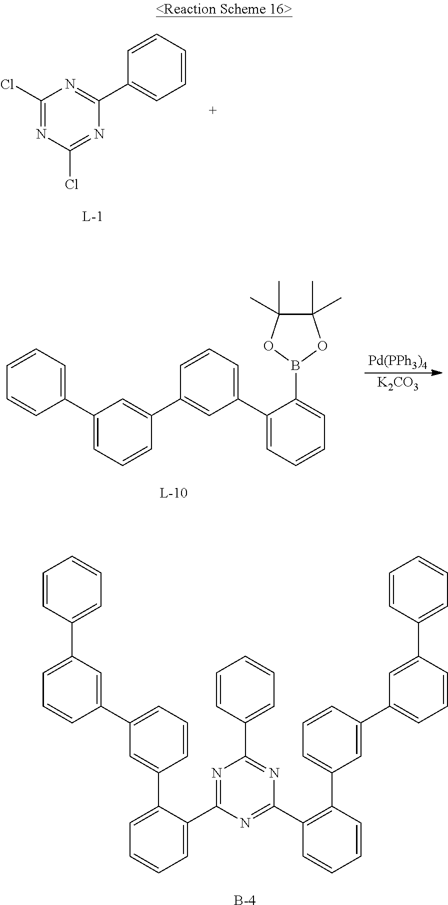

5.0 g (22.12 mmol) of the intermediate L-1 (2,4-dichloro-6-phenyl-s-triazine), 21.99 g (46.60 mmol) of the intermediate L-10, 7.64 g (55.30 mmol) of potassium carbonate, and 1.28 g (1.11 mmol) of Pd(PPh₃)₄ (Tetrakis(triphenyl phosphine)palladium (0)) were added to 100 mL of tetrahydrofuran and 30 mL of water in a 250 mL flask, and the mixture was heated and refluxed for 10 hours for a nitrogen stream. Then, a solid crystallized by adding the obtained mixture to 500 mL of methanol was filtered, dissolved in monochlorobenzene, filtered again with silica gel/Celite and then, recrystallized with methanol after removing the organic solvent in an appropriate amount, obtaining a compound B-4 (13.2 g, 78% of a yield). The element analysis result of the compound B-4 was provided as follows.

calcd. C₅₇H₃₉N₃: C, 89.38; H, 5.13; N, 5.49; found: C, 89.77; H, 5.36; N, 5.14;

### Synthesis Example 8: Synthesis of Compound A-34

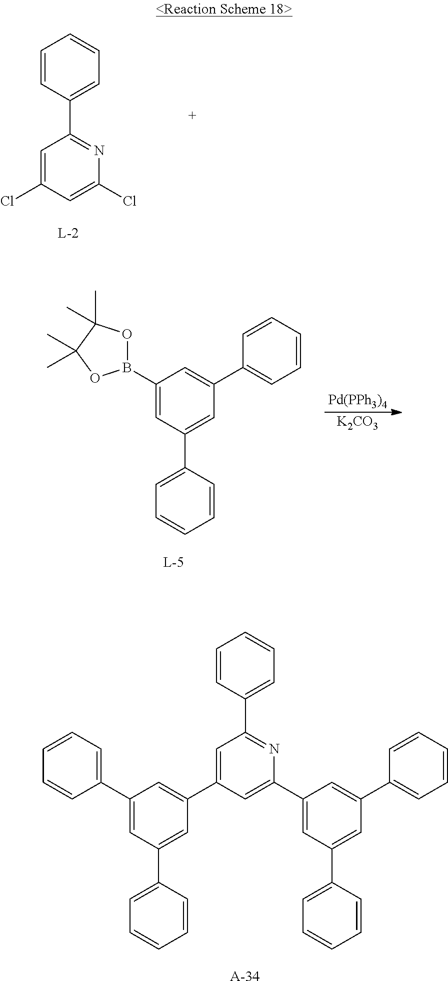

5.0 g (22.31 mmol) of the intermediate L-2 (2,4-dichloro-6-phenyl-pyridine), 18.28 g (51.32 mmol) of the intermediate L-5, 7.71 g (55.78 mmol) of potassium carbonate, and 1.29 g (1.12 mmol) of Pd(PPh₃)₄ (tetrakis(triphenylphosphine)palladium (0)) were added to 100 mL of tetrahydrofuran and 30 mL of water in a 250 mL flask, and the mixture was heated and refluxed for 10 hours under a nitrogen stream. Then, a solid crystallized by adding the obtained mixture to 500 mL of methanol was filtered, dissolved in monochlorobenzene, filtered again with silica gel/Celite, and then, recrystallized with methanol after removing the organic solvent in an appropriate amount, obtaining a compound A-34 (9.65 g, 71% of a yield). The element analysis result of the compound A-34 was provided as follows.

calcd. C₄₇H₃₃N: C, 92.27; H, 5.44; N, 2.29; found: C, 92.12; H, 5.23; N, 2.26;

### Synthesis Example 9: Synthesis of Compound A-36

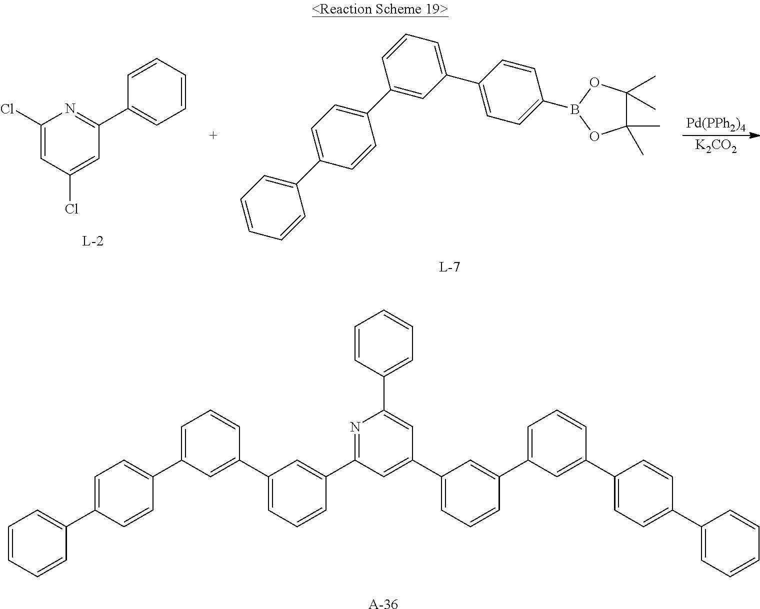

5.0 g (22.31 mmol) of the intermediate L-2, 22.19 g (51.32 mmol) of the intermediate L-7, 7.71 g (55.78 mmol) of potassium carbonate, and 1.29 g (1.12 mmol) of Pd(PPh₃)₄ (tetrakis(triphenyl phosphine)palladium (0)) were added to 100 mL of tetrahydrofuran and 30 mL of water in a 250 mL flask, and the mixture was heated and refluxed for 10 hours under a nitrogen stream. Then, a solid crystallized by adding the obtained mixture to 500 mL of methanol was filtered, dissolved in monochlorobenzene, filtered again with silica gel/Celite and then, recrystallized with methanol after removing the organic solvent in an appropriate amount, obtaining a compound A-36 (14.3 g, 84% of a yield). The element analysis result of the compound A-36 was provided as follows.

calcd. C₅₉H₄₁N: C, 92.76; H, 5.41; N, 1.83; found: C, 92.66; H, 5.23; N, 1.75;

### Synthesis Example 10: Synthesis of Compound A-55

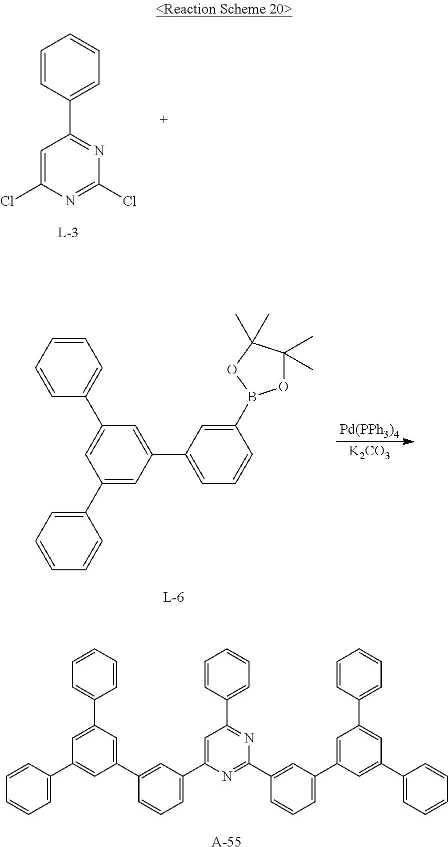 [SMILES: Clc1cc(-c2ccccc2)nc(Cl)n1 ; CC1(C)OB(c2cccc(-c3cc(-c4ccccc4)cc(-c4ccccc4)c3)c2)OC1(C)C ; "K2CO3" ; c1ccc(-c2cc(-c3ccccc3)cc(-c3cccc(-c4cc(-c5ccccc5)nc(-c5cccc(-c6cc(-c7ccccc7)cc(-c7ccccc7)c6)c5)n4)c3)c2)cc1 ; "A-55" ; "Pd(PPh3)4"]

5.0 g (22.22 mmol) of the intermediate L-3, 22.09 g (51.1 mmol) of the intermediate L-6, 7.68 g (55.54 mmol) of potassium carbonate, and 1.28 g (1.11 mmol) of Pd(PPh₃)₄ (tetrakis(triphenylphosphine)palladium (0)) were added to 100 mL of tetrahydrofuran and 30 mL of water in a 250 mL flask, and the mixture was heated and refluxed for 10 hours under a nitrogen stream. Then, a solid crystallized by adding the obtained mixture to 500 mL of methanol was filtered, dissolved in monochlorobenzene, filtered again with silica gel/Celite and then, recrystallized with methanol after removing the organic solvent in an appropriate amount, obtaining a compound A-55 (12.68 g, 75% of a yield). The element analysis result of the compound A-55 was provided as follows.

calcd. C₅₈H₄₀N₂: C, 91.07; H, 5.27; N, 3.66; found: C, 91.12; H, 5.17; N, 3.56;

### Synthesis Example 11: Synthesis of Compound A-54

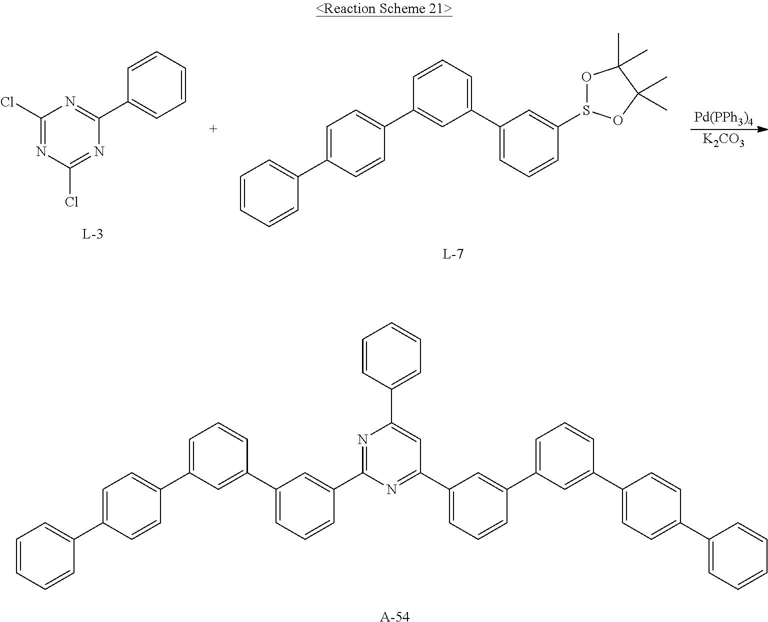

5.0 g (22.22 mmol) of the intermediate L-3, 22.09 g (51.1 mmol) of the intermediate L-7, 7.68 g (55.54 mmol) of potassium carbonate, and 1.28 g (1.11 mmol) of Pd(PPh₃)₄ (tetrakis(triphenylphosphine) palladium (0)) were added to 100 mL of tetrahydrofuran and 30 mL of water in a 250 mL flask, and the mixture was heated and refluxed for 10 hours under a nitrogen stream. Then, a solid crystallized by adding the obtained mixture to 500 mL of methanol was filtered, dissolved in monochlorobenzene, filtered again with silica gel/Celite and then, recrystallized with methanol after removing the organic solvent in an appropriate amount, obtaining a compound A-54 (13.12 g, 77% of a yield). The element analysis result of the compound A-54 was provided as follows.

calcd. C₅₈H₄₀N₂: C, 91.07; H, 5.27; N, 3.66; found: C, 91.01; H, 5.12; N, 3.48;

### Comparative Example 1: Synthesis of CBP

A compound represented by the following Chemical Formula a was synthesized according to the same method as a method described in International Publication No. WO 2013032035.

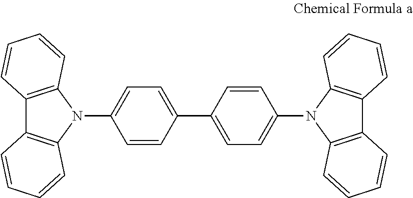 [SMILES: c1ccc2c(c1)c1ccccc1n2-c1ccc(-c2ccc(-n3c4ccccc4c4ccccc43)cc2)cc1]

### (Simulation Characteristics Comparison of Prepared Compounds)

Energy level of each material was calculated in a Gaussian 09 method by using a supercomputer GAIA (IBM power 6), and the result is provided in the following Table 1.

TABLE 1

| | Com- | HOMO | LUMO | T1 | S1 |
|---|---|---|---|---|---|
| Examples | pound | (eV) | (eV) | (eV) | (eV) |
| Comparative | CBP | −5.319 | −1.231 | 2.971 | 3.560 |
| Example 1 | | | | | |
| Synthesis | A-1 | −6.027 | −1.868 | 2.94 | 3.728 |
| Example 1 | | | | | |
| Synthesis | A-2 | −6.041 | −1.874 | 2.883 | 3.629 |
| Example 2 | | | | | |
| Synthesis | A-5 | −6.02 | −1.871 | 2.93 | 3.737 |
| Example 3 | | | | | |
| Synthesis | A-7 | −6.037 | −1.922 | 2.784 | 3.689 |
| Example 4 | | | | | |
| Synthesis | A-25 | −5.965 | −1.819 | 2.941 | 3.748 |
| Example 5 | | | | | |
| Synthesis | B-5 | −5.938 | −1.7 | 3.067 | 3.638 |
| Example 6 | | | | | |
| Synthesis | B-4 | −5.902 | −1.682 | 3.062 | 3.653 |
| Example 7 | | | | | |
| — | B-13 | −5.753 | −1.687 | 2.942 | 3.486 |
| — | A-51 | −6.025 | −1.694 | 2.934 | 3.828 |
| — | A-52 | −5.905 | −1.706 | 2.87 | 3.734 |
| Synthesis | A-55 | −5.922 | −1.713 | 2.905 | 3.769 |
| Example 10 | | | | | |
| Synthesis | A-54 | −5.722 | −1.714 | 2.907 | 3.672 |
| Example 11 | | | | | |

As shown in Table 1, since a compound showed electron-transporting characteristics when it had a HOMO ranging from −5.0 eV to −6.2 eV and a LUMO ranging from −1.65 eV to −2.1 eV as a desired HOMO/LUMO energy level in a simulation, Comparative Example 1 satisfied the HOMO level but not the LUMO level and thus, unbalance between holes and electrons was expected compared with the compounds A-1, A-2, A-5, A-7, A-25, B-4, B-5, B-13, A-51, A-52, A-54 and 55.

The compound of the present invention had an appropriate energy level compared with Comparative Example 1 and was expected to show excellent efficiency and life-span.

### Manufacture of Organic Light Emitting Diode (Device Including Electron Transport Auxiliary Layer)

### Device Example 1

A glass substrate coated with ITO (indium tin oxide) to be 1500 Å thick was ultrasonic wave-washed with a distilled water. Subsequently, the glass substrate was ultrasonic wave-washed with a solvent such as isopropyl alcohol, acetone, methanol, and the like, moved to a plasma cleaner, cleaned by using oxygen plasma for 10 minutes, and then, moved to a vacuum depositor. This obtained ITO transparent electrode was used as an anode, and HT13 was vacuum-deposited on the ITO substrate to form 1400 Å-thick hole injection layer. A 200 Å-thick emission layer was formed thereon by vacuum-depositing 9,10-di(2-naphthyl)anthracene (ADN) as a blue fluorescent light emitting host doped with 5 wt % of 9,10-di(2-naphthyl)anthracene (ADN) and BD01 as a dopant. The structures of AND and BD01 are shown below. A-1 of Synthesis Example 1 was vacuum-deposited on the emission layer to form a 50 Å-thick electron transport auxiliary layer. Tris(8-hydroxyquinoline)aluminum (Alq3) was vacuum-deposited on the electron transport auxiliary layer to form a 310 Å-thick electron transport layer (ETL), and Liq (15 Å) and Al (1200 Å) were sequentially vacuum-deposited on the electron transport layer (ETL) to form a cathode, manufacturing an organic light emitting diode.

The organic light emitting diode had a five-layered organic thin film structure and specifically,

ITO/HT13 1400 Å/EML[ADN:BD01=95:5 wt %] 200 Å/compound A-1 50 Å/Alq3 310 Å/Liq 15 Å/Al 1200 Å.

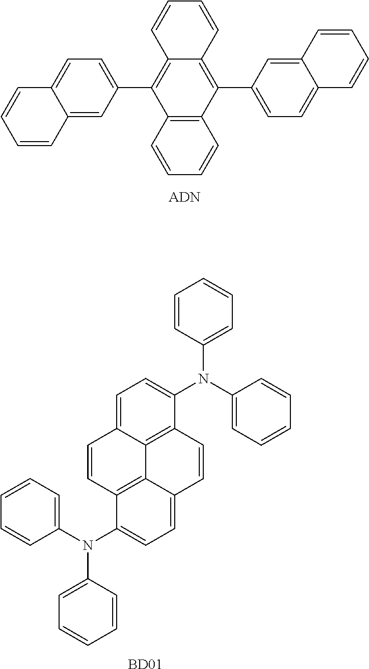

### Device Example 2

An organic light emitting diode was manufactured according to the same method as Example 1 except for using A-2 of Synthesis Example 2 instead of A-1 of Synthesis Example 1.

### Device Example 3

An organic light emitting diode was manufactured according to the same method as Example 1 except for using A-5 of Synthesis Example 3 instead of A-1 of Synthesis Example 1.

### Device Example 4

An organic light emitting diode was manufactured according to the same method as Example 1 except for using A-7 of Synthesis Example 4 instead of A-1 of Synthesis Example 1.

### Device Example 5

An organic light emitting diode was manufactured according to the same method as Example 1 except for using A-25 of Synthesis Example 5 instead of A-1 of Synthesis Example 1.

### Device Comparative Example 1

An organic light emitting diode was manufactured according to the same method as Example 1 except for not using the electron transport auxiliary layer.

### Evaluation

Current density and luminance changes depending on a voltage, luminous efficiency and life-span of each organic light emitting diode according to Device Examples 1, 2, 3, 4, 5 and Device Comparative Example 1 were measured.

Specific measurement methods were as follows, and the results were provided in Table 1.

(1) Measurement of Current Density Change Depending on Voltage Change

The obtained organic light emitting diodes were measured for current value flowing in the unit device while increasing the voltage from 0 V to 10 V using a current-voltage meter (Keithley 2400), the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

Current efficiency (cd/A) at the same current density (10 mA/cm²) were calculated by using the luminance, current density, and voltages (V) from the items (1) and (2).

(4) Life-Span

T97 life-spans of the organic light emitting diodes of Example 1 and Comparative Example 1 were measured as a time when their luminance decreased down to 97% relative to the initial luminance (cd/m²) after emitting light with 750 cd/m² as the initial luminance (cd/m²) and measuring their luminance decrease depending on time with a Polanonix life-span measurement system.

TABLE 2

| | Electron | | | | |
|---|---|---|---|---|---|
| | transport | | | Color | T97 life- |
| | auxiliary | Driving | Luminous | coordinate | span(h) |
| Device | layer | voltage | efficiency | (x, y) | @750 nit |
| Device | com- | 5.04 | 6.2 | (0.133, | 180 |
| Example 1 | poundA-1 | | | 0.149) | |
| Device | com- | 5.18 | 6.8 | (0.133, | 175 |
| Example 2 | poundA-2 | | | 0.149) | |
| Device | com- | 5.10 | 6.7 | (0.133, | 170 |
| Example 3 | poundA-5 | | | 0.149) | |
| Device | com- | 5.15 | 6.6 | (0.133, | 200 |
| Example 4 | poundA-7 | | | 0.149) | |
| Device | com- | 5.15 | 6.5 | (0.133, | 180 |
| Example 5 | poundA-25 | | | 0.149) | |
| Device | Not used | 5 | 6.8 | (0.133, | 120 |
| Comparative | | | | 0.146) | |
| Example 1 | | | | | |

Referring to Table 2, the organic light emitting diode according to Device Example 4 showed about 1.7 times increased life-span compared with that of the organic light emitting diode according to Device Comparative Example 1, and the organic light emitting diodes according to Device Examples 1, 2, 3 and 5 showed about 1.5 times increased life-span compared with that of the organic light emitting diode according to Device Comparative Example 1. Accordingly, the electron-transporting auxiliary layer turned out to improve life-span characteristics of an organic light emitting diode.

### Manufacture of Organic Light Emitting Diode (Device Using Compounds as Host)

### Device Comparative Example 2

Specifically illustrating a method of manufacturing an organic light-emitting device, a anode is manufactured by cutting an ITO glass substrate having sheet resistance of 15 Ω/cm² into a size of 50 mm×50 mm×0.7 mm, respectively washing the cut substrate with an ultrasonic wave in acetone, isopropyl alcohol, and pure water for 15 minutes, and then, cleaning it with an UV ozone for 30 minutes.

Subsequently, the following HTM compound was vacuum-deposited to form a 1200 Å-thick hole injection layer on this ITO transparent electrode as a 1000 Å-thick anode.

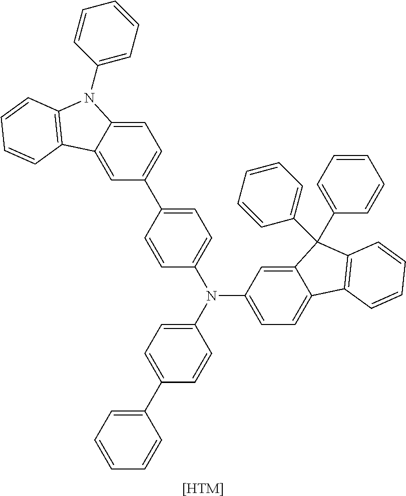

4,4-N,N-dicarbazolebiphenyl (CBP) as a host of an emission layer doped with 7 wt % of the following PhGD compound as a phosphorescent green dopant was vacuum-deposited to form a 300 Å-thick emission layer.

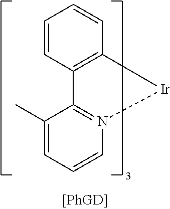

Subsequently, BAlq [bis(2-methyl-8-quinolinolato-N1,08)-(1,1′-Biphenyl-4-olato)aluminum] was laminated to be 50 Å-thick, and Alq3 [Tris(8-hydroxyquinolinato)aluminium] was sequentially laminated to be 250 Å thick to form an electron transport layer on the emission layer.

On the electron transport layer, LiF and Al were sequentially vacuum-deposition to respectively be 5 Å thick and 1000 Å thick to form a cathode, manufacturing an organic light emitting device.

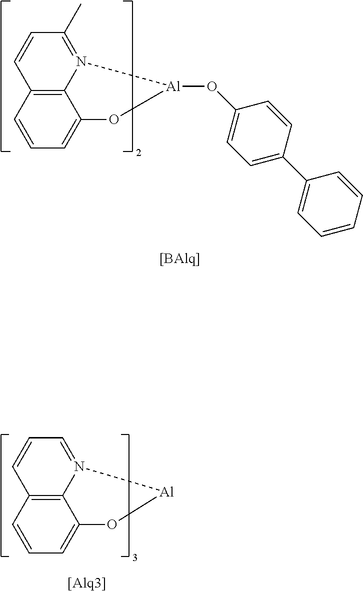

### Device Example 6

An organic light emitting device was manufactured according to the same method as the Device Comparative Example 2 except for using the compound A-1 according to Synthesis Example 1 as a host of an emission layer.

### Device Example 7

An organic light emitting device was manufactured according to the same method as the Device Comparative Example 2 except for using the compound A-2 according to Synthesis Example 2 as a host of an emission layer.

### Device Example 8

An organic light emitting device was manufactured according to the same method as the Device Comparative Example 2 except for using the compound A-5 according to Synthesis Example 3 as a host of an emission layer.

### Device Example 9

An organic light emitting device was manufactured according to the same method as the Device Comparative Example 2 except for using the compound A-7 according to Synthesis Example 4 as a host of an emission layer.

### Device Example 10

An organic light emitting device was manufactured according to the same method as the Device Comparative Example 2 except for using the compound A-25 according to Synthesis Example 5 as a host of an emission layer.

### (Performance Measurement of Organic Light Emitting Diode)

Current density change, luminance change, and luminous efficiency of each organic light emitting diode according to Device Examples 6, 7, 8, 9 and 10 and Device Comparative Example 2 were measured.

Specific measurement methods are as follows, and the results are shown in the following Table 3.

(1) Measurement of Current Density Change Depending on Voltage Change

The obtained organic light emitting diodes were measured for current value flowing in the unit device while increasing the voltage from 0 V to 10 V using a current-voltage meter (Keithley 2400), and the measured current value was divided by area to provide the result.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

Current efficiency (cd/A) at the same current density (10 mA/cm²) were calculated by using the luminance, current density, and voltages (V) from the items (1) and (2).

(4) Life-Span

A time when current efficiency (cd/A) was decreased to 90% was measured while maintaining luminance (cd/m²) to be 5000 cd/m².

TABLE 3

| | Emission | Driving | Color | | 90% life- |
|---|---|---|---|---|---|
| | layer | voltage | (EL | Efficiency | span (h) At |
| Nos. | (host) | (V) | color) | (cd/A) | 5000 cd/m² |
| Device | A-1 | 4.06 | Green | 58.1 | 360 |
| Example 6 | | | | | |
| Device | A-2 | 4.08 | Green | 57.6 | 240 |
| Example 7 | | | | | |
| Device | A-5 | 4.28 | Green | 50.4 | 380 |
| Example 8 | | | | | |
| Device | A-7 | 4.35 | Green | 50.7 | 450 |
| Example 9 | | | | | |
| Device | A-25 | 4.14 | Green | 54.2 | 440 |
| Example 10 | | | | | |
| Device | CBP | 6.70 | Green | 34.8 | 50 |
| Comparative | | | | | |
| Example 2 | | | | | |

As shown in Table 3, when the compound according to the present invention was used as a host for an emission layer, a device showed a driving voltage of early 4V overall, which was moved up compared with that of Device Comparative Example 2 and about 1.5 times increased luminous efficiency compared with that of Device Comparative Example 2. In addition, the compound according to the present invention showed improved characteristics and thus, a long life-span compared with Device Comparative Example 2. In other words, the compound showed improved characteristics in terms of a driving voltage, luminous efficiency and/or power efficiency.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims. Therefore, the aforementioned embodiments should be understood to be exemplary but not limiting the present invention in any way.

[Description of Symbols]

| | |
|---|---|
| 100: organic light emitting diode | 200: organic light emitting diode |
| 105: organic layer | |
| 110: cathode | |
| 120: anode | |
| 130: emission layer | 230: emission layer |
| 140: hole auxiliary layer | |

1. A compound represented by the following Chemical Formula 1: 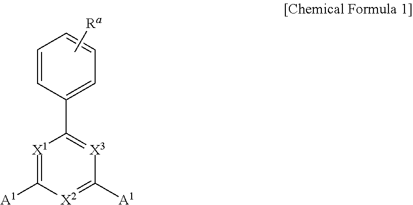
wherein, in Chemical Formula 1,
X¹ to X³ are N,
R^{a} is hydrogen, deuterium, or a substituted or unsubstituted C1 to C10 alkyl group, and
A¹ is represented by Chemical Formula I or II, 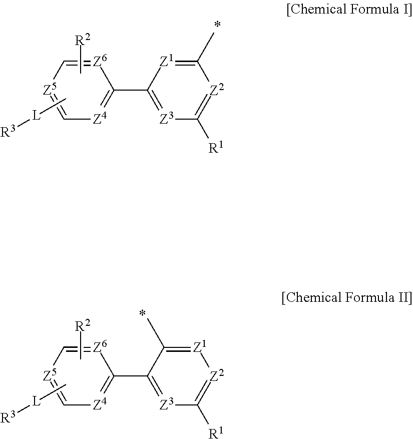
wherein, in Chemical Formulae I and II,
Z¹ to Z⁶ are independently C or CR^{c},
R¹, R², and R^{c} are each independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C6 to C30 arylamine group, a substituted or unsubstituted C1 to C30 alkoxy group, a substituted or unsubstituted C3 to C40 silyl group, or a combination thereof,
L is a single bond, or a C6 to C30 arylene group,
R³ is hydrogen, or a substituted or unsubstituted C6 to C30 aryl group, provided that, when the L is a single bond, at least one of R¹ to R³ is not hydrogen, and
* is a linking point,
wherein “substituted” refers to that at least one hydrogen is replaced by deuterium, a halogen, a hydroxy group, an amino group, a C1 to C30 alkyl group, or a C6 to C30 aryl group.

2. The compound of claim 1, wherein Chemical Formula 1 is represented by one of Chemical Formulae I-a, I-b, I-c, II-a, II-b, or II-c: 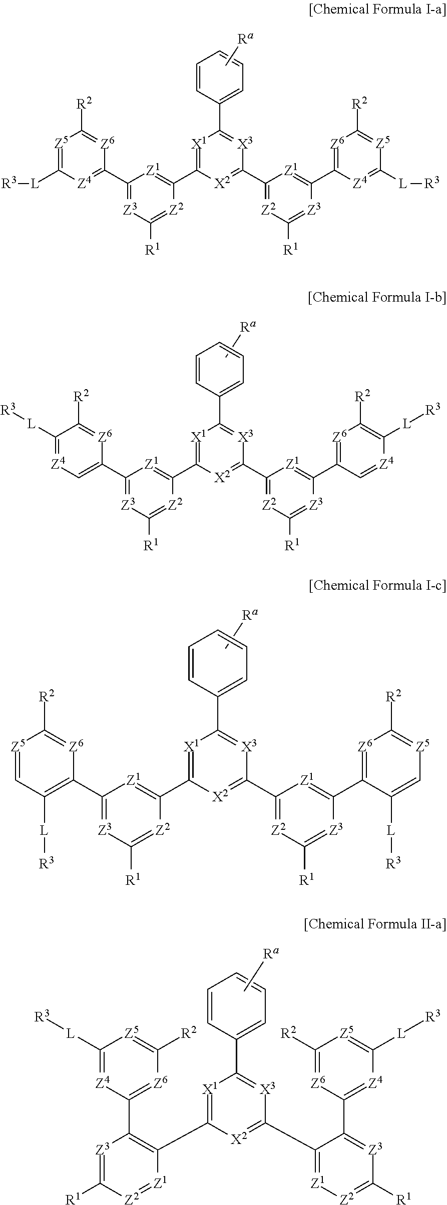 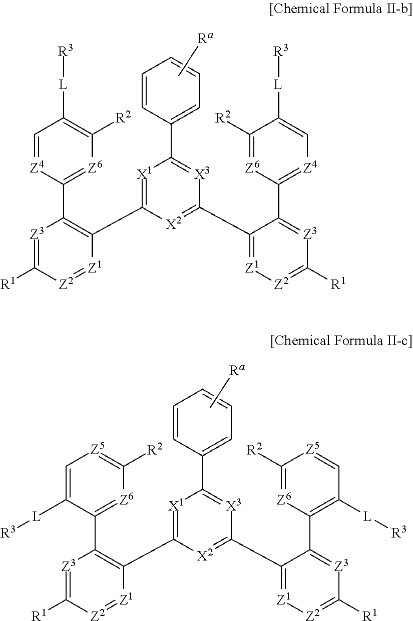
wherein, in Chemical Formulae I-a, I-b, I-c, II-a, II-b, and II-c,
X¹ to X³ are N,
R^{a} is hydrogen, deuterium, or a substituted or unsubstituted C1 to C10 alkyl group,
Z¹ to Z⁶ are independently CR^{c},
R¹, R², and R^{c} are each independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C6 to C30 arylamine group, a substituted or unsubstituted C1 to C30 alkoxy group, a substituted or unsubstituted C3 to C40 silyl group, or a combination thereof,
L is a single bond, or a C6 to C30 arylene group,
R³ is hydrogen, or a substituted or unsubstituted C6 to C30 aryl group, provided that, when the L is a single bond, at least one of R¹ to R³ is not hydrogen, and
wherein “substituted” refers to that at least one hydrogen is replaced by deuterium, a halogen, a hydroxy group, an amino group, a C1 to C30 alkyl group, or a C6 to C30 aryl group.

3. The compound of claim 1, wherein Chemical Formula 1 is represented by one of Chemical Formulae I-d or II-d: 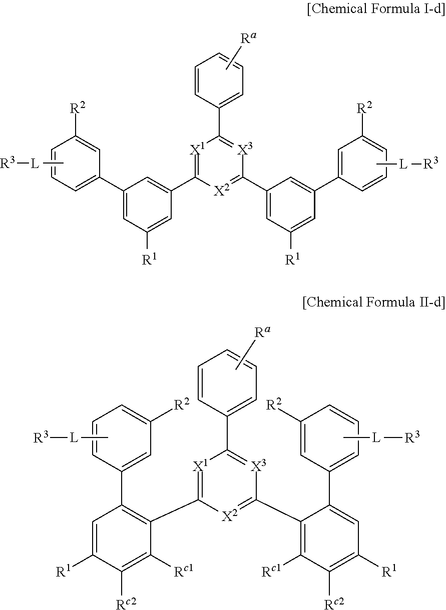
wherein, in Chemical Formulae I-d and II-d,
X¹ to X³ are independently N,
R^{a} is hydrogen, deuterium, or a substituted or unsubstituted C1 to C10 alkyl group,
R¹, R^{c1}, R^{c2}, and R² are each independently hydrogen, deuterium, a substituted or unsubstituted C1 to C30 alkyl group, a substituted or unsubstituted C3 to C30 cycloalkyl group, a substituted or unsubstituted C6 to C30 aryl group, a substituted or unsubstituted C6 to C30 arylamine group, a substituted or unsubstituted C1 to C30 alkoxy group, a substituted or unsubstituted C3 to C40 silyl group, or a combination thereof,
L is a single bond, or a C6 to C30 arylene group,
R³ is hydrogen, or a substituted or unsubstituted C6 to C30 aryl group, provided that, when the L is a single bond, at least one of R′ to R³ is not hydrogen, and
wherein “substituted” refers to that at least one hydrogen is replaced by deuterium, a halogen, a hydroxy group, an amino group, a C1 to C30 alkyl group, or a C6 to C30 aryl group.

4. (canceled)

5. The compound of claim 1, wherein R¹, R², and R^{c} are each independently hydrogen, a substituted or unsubstituted phenyl group, a substituted or unsubstituted biphenyl group, a substituted or unsubstituted terphenyl group, or a substituted or unsubstituted naphthyl group, and
R³ is hydrogen, a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted triphenylene group, or a combination thereof.

6. The compound of claim 1, wherein R³ is selected from substituted or unsubstituted groups of Group I: 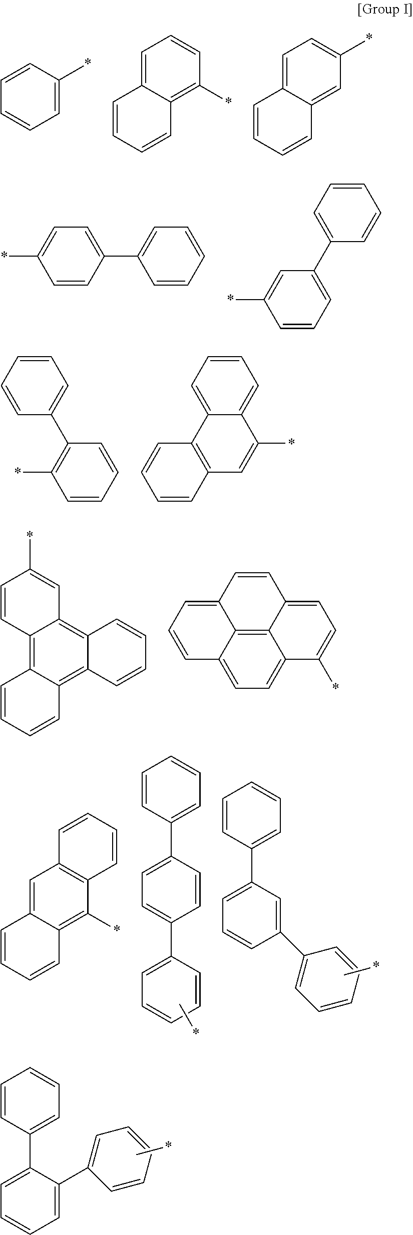
wherein, in Group I,
* is a linking point,
wherein “substituted” refers to that at least one hydrogen is replaced by deuterium, a halogen, a hydroxy group, an amino group, a C1 to C30 alkyl group, or a C6 to C30 aryl group.

7. The compound of claim 1, wherein L is a single bond, a substituted or unsubstituted phenylene group, a substituted or unsubstituted biphenylene group, a substituted or unsubstituted naphthyl group, or a combination thereof,
wherein “substituted” refers to that at least one hydrogen is replaced by deuterium, a halogen, a hydroxy group, an amino group, a C1 to C30 alkyl group, or a C6 to C30 aryl group.

8. The compound of claim 1, wherein L is a single bond, or selected from substituted or unsubstituted groups of Group II: 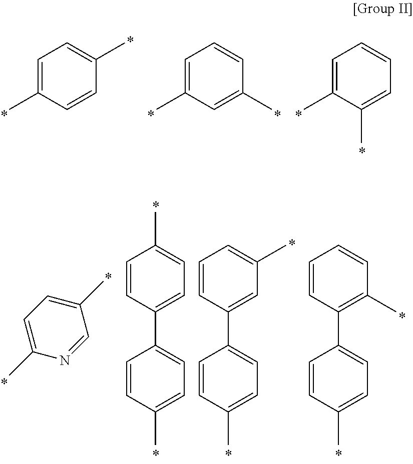
wherein, in Group II,
* is a linking point,
wherein “substituted” refers to that at least one hydrogen is replaced by deuterium, a halogen, a hydroxy group, an amino group, a C1 to C30 alkyl group, or a C6 to C30 aryl group.

9. The compound of claim 1, wherein the compound represented by Chemical Formula 1 is selected from Chemical Formulae A-1 to A-15, A-25, A-29, A-30, A-49, A-50, and B-1 to B-12:

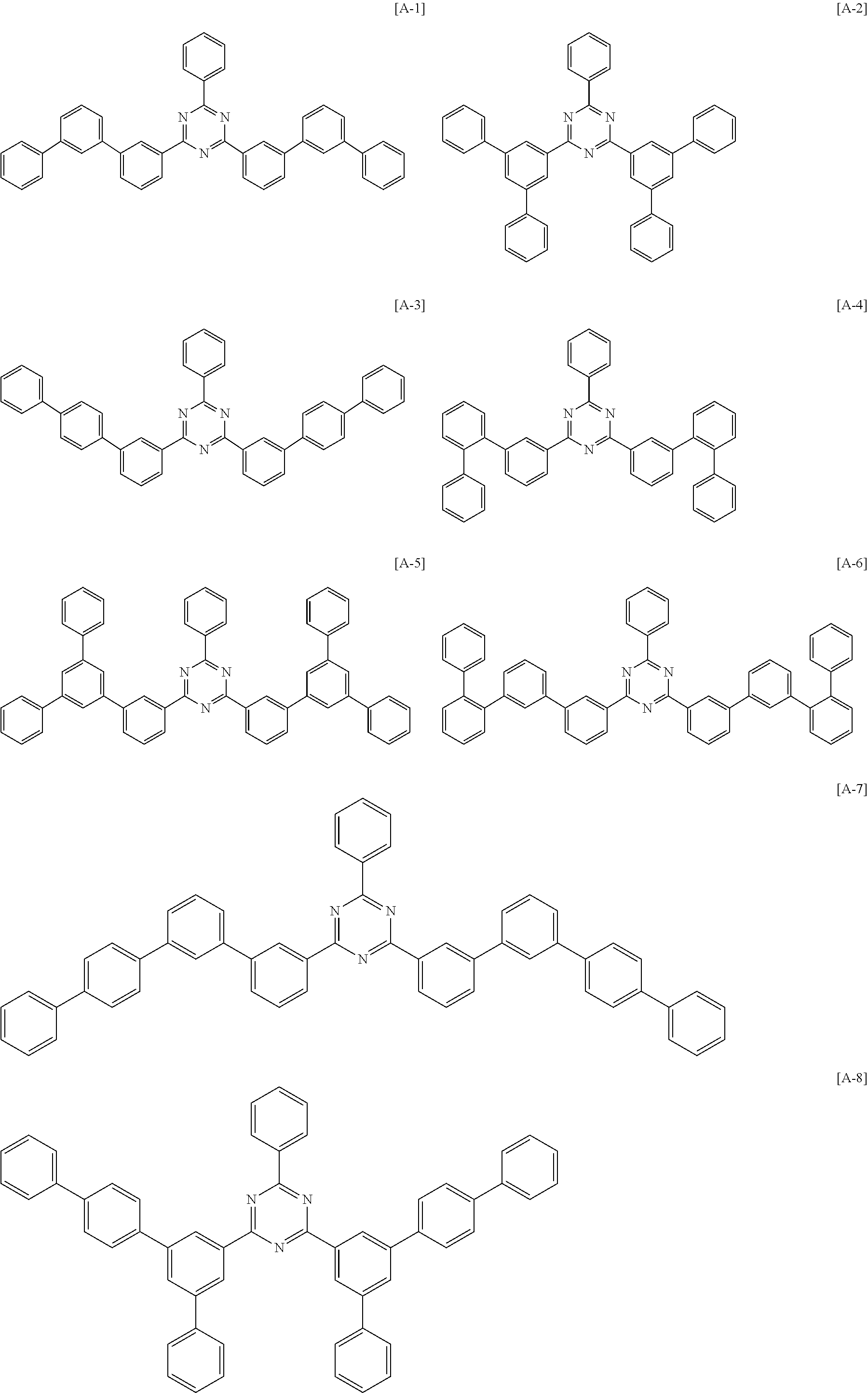

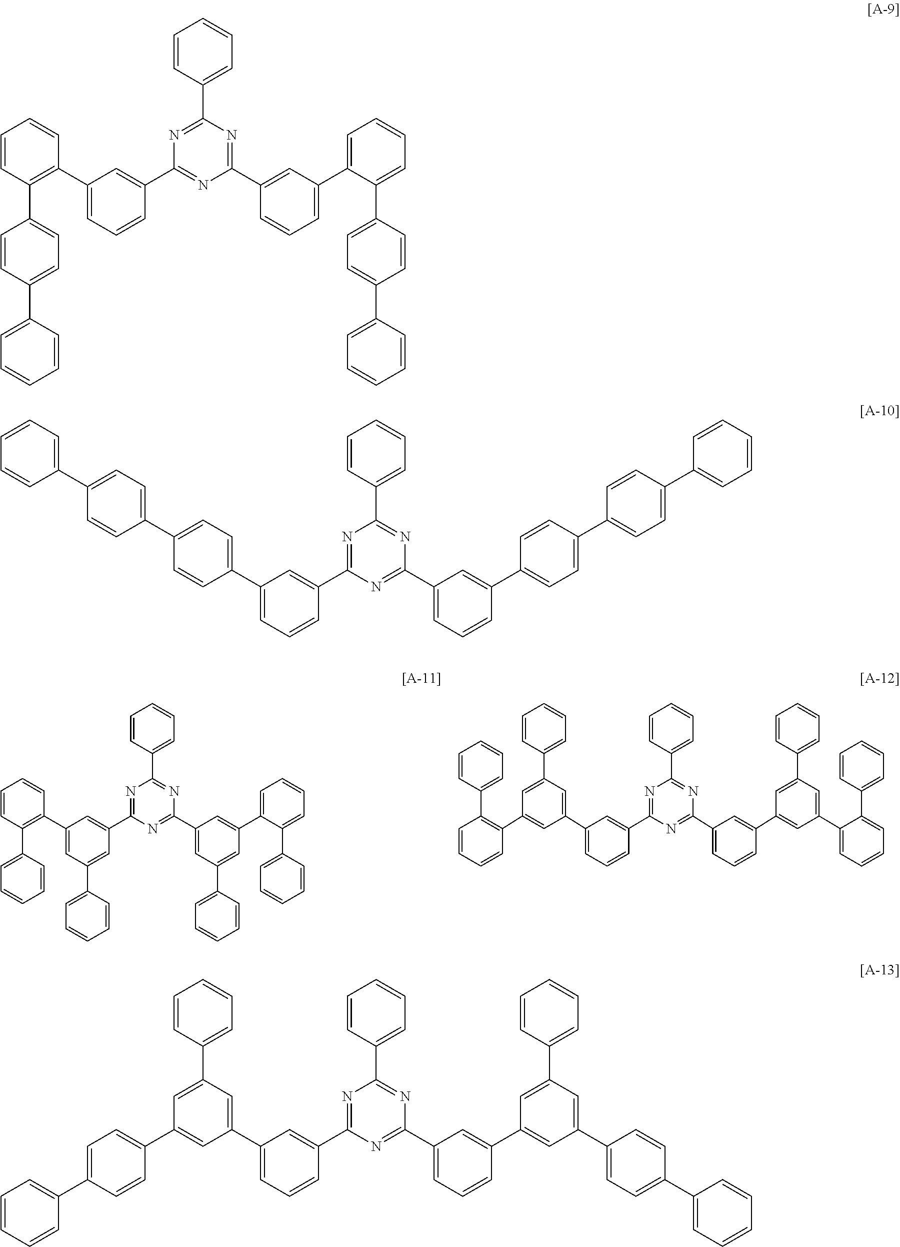

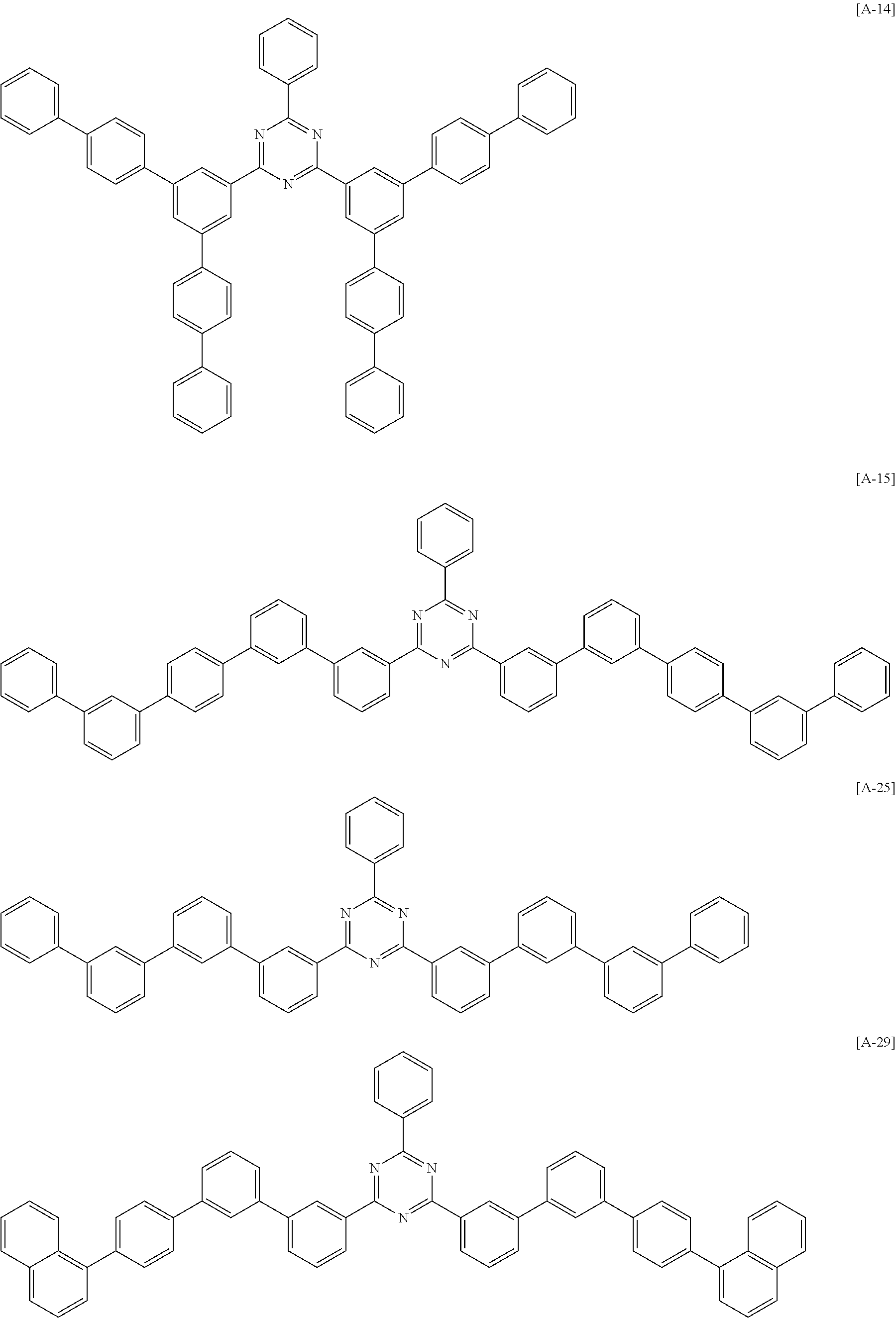

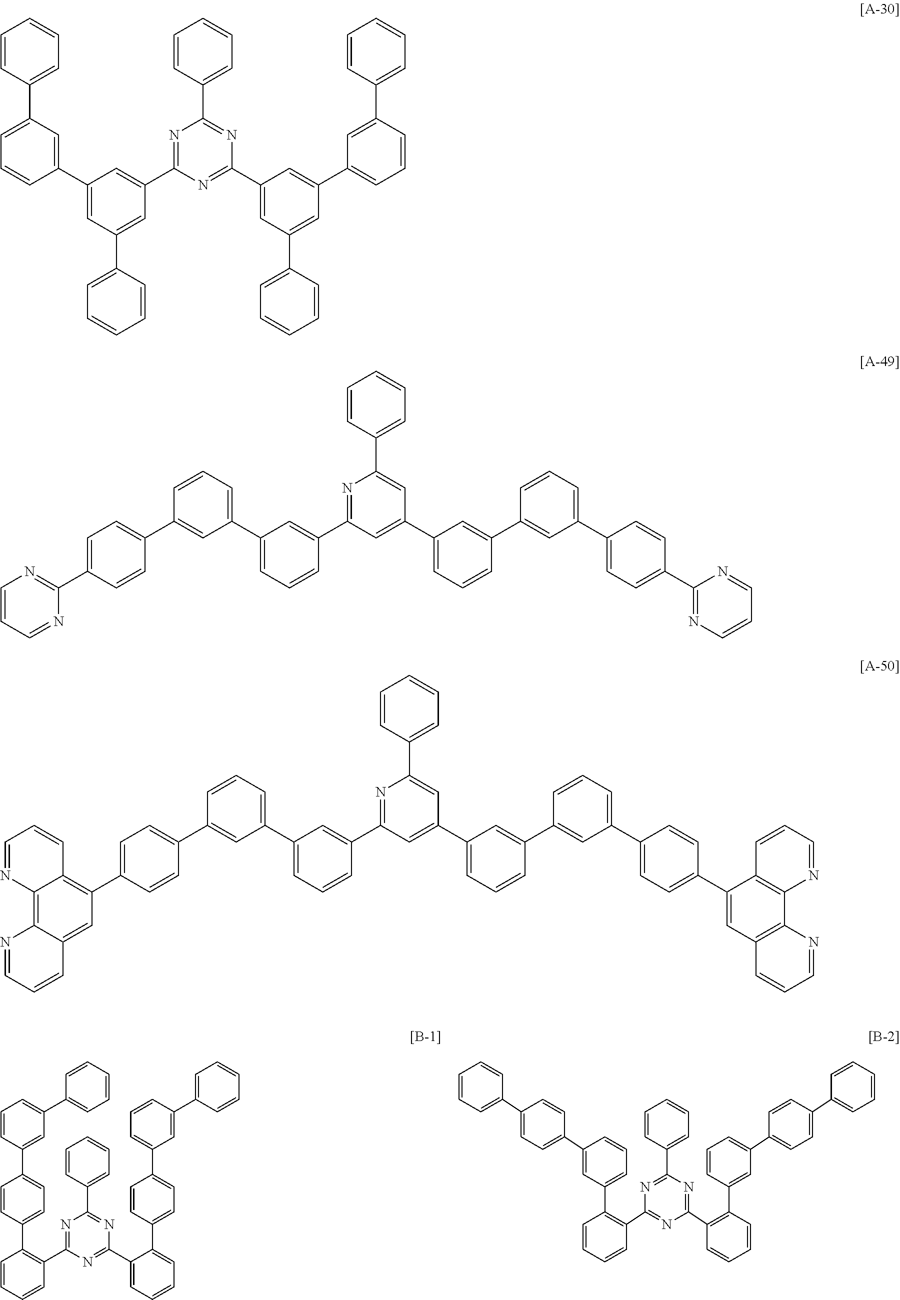

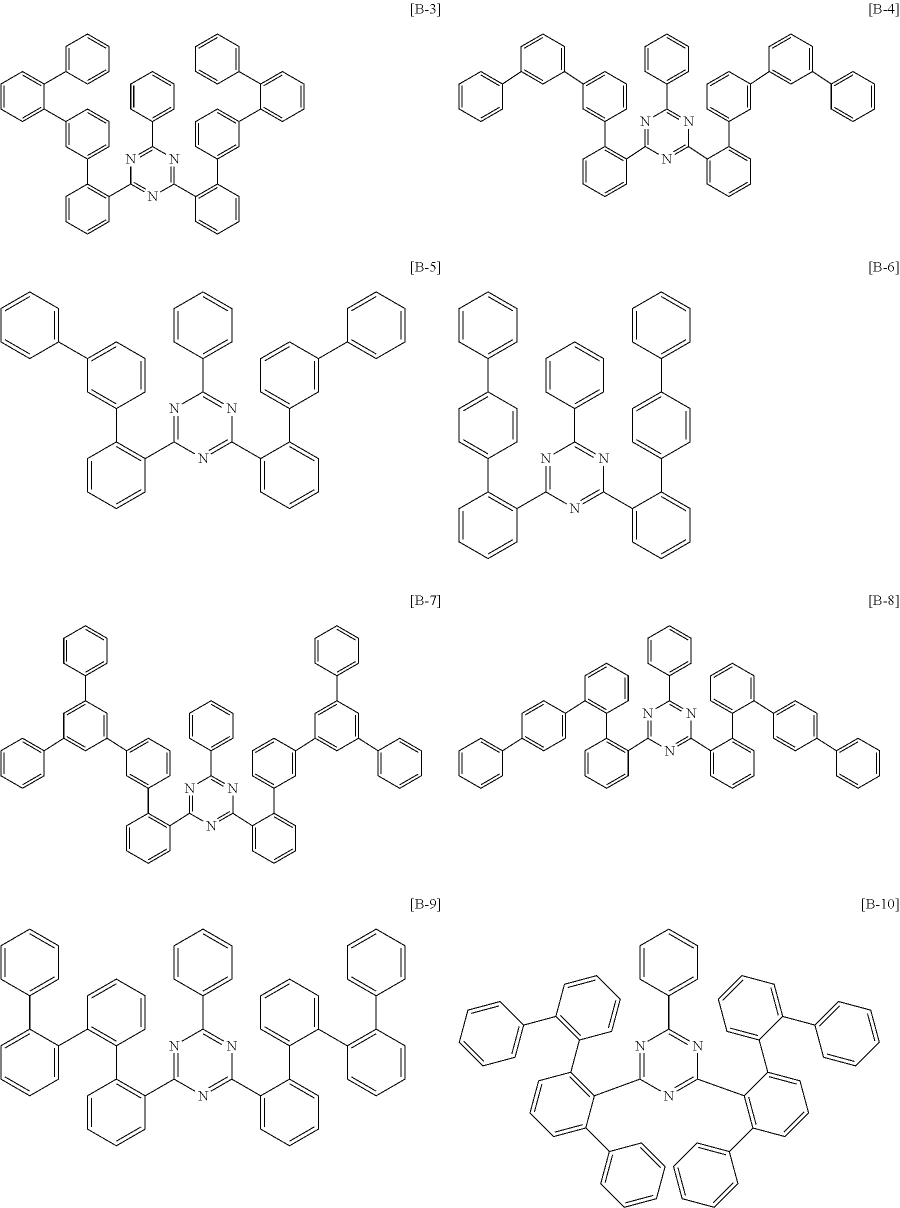

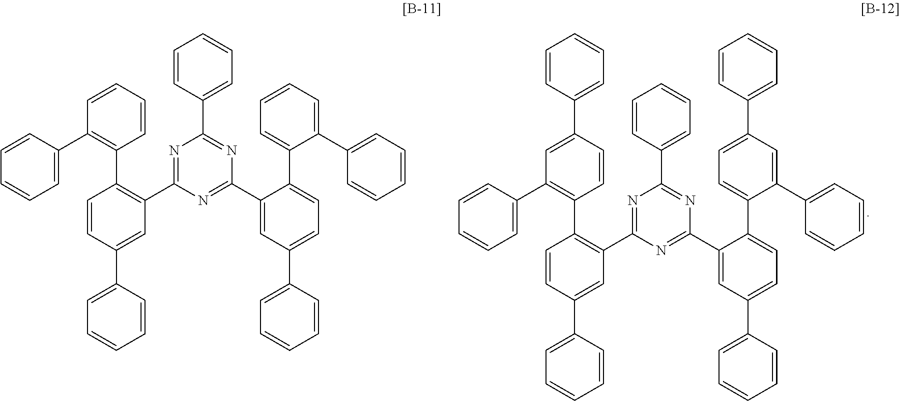 [SMILES: c1ccc(-c2ccc(-c3ccccc3-c3ccccc3)c(-c3nc(-c4ccccc4)nc(-c4cc(-c5ccccc5)ccc4-c4ccccc4-c4ccccc4)n3)c2)cc1 ; c1ccc(-c2ccc(-c3ccc(-c4ccccc4)cc3-c3nc(-c4ccccc4)nc(-c4cc(-c5ccccc5)ccc4-c4ccc(-c5ccccc5)cc4-c4ccccc4)n3)c(-c3ccccc3)c2)cc1]

10. The compound of claim 1, wherein the compound is used for an organic optoelectric device.

11. An organic optoelectric device, comprising:
an anode and a cathode facing each other; and
at least one organic layer between the anode and the cathode, wherein:
the organic layer includes the compound of claim 1.

12. The organic optoelectric device of claim 11, wherein:
the organic layer is an emission layer, and
the emission layer includes the compound.

13. The organic optoelectric device of claim 12, wherein the compound is included as a host of the emission layer.

14. The organic optoelectric device of claim 11, wherein the organic layer includes at least one auxiliary layer selected from a hole injection layer (HIL), a hole transport layer (HTL), a hole transport auxiliary layer, an electron transport auxiliary layer, an electron transport layer (ETL), and an electron injection layer (EIL), and
the auxiliary layer includes the compound.

15. A display device comprising the organic optoelectric device of claim 11.
